(19) United States
(12) Patent Application Publication
(10) Pub. No.: US 2011/0183999 A1
(43) Pub. Date: Jul. 28, 2011

(54) NOVEL POLYMORPHIC FORMS OF METHYL CARBAMATE

(75) Inventors: Alfons GRUNENBERG, Dormagen (DE); Franz-Josef MAIS, Dusseldorf (DE); Winfried JOENTGEN, Koln (DE); Birgit KEIL, Dusseldorf (DE)

(73) Assignee: BAYER SCHERING PHARMA AKTIENGESELLSCHAFT, Berlin (DE)

(21) Appl. No.: 12/954,961

(22) Filed: Nov. 29, 2010

(30) Foreign Application Priority Data
Nov. 27, 2009 (EP) 09177373.9
Dec. 3, 2009 (EP) 09177908.2

(51) Int. Cl. A61K 31/506; C07D 401/14; A61P 9/00

(52) U.S. Cl. 514/256; 544/322

(57) ABSTRACT

The invention relates to novel forms of methyl {4,6-diamino-2-[1-(2-fluorobenzyl)-1H-pyrazolo[3,4-b]pyridin-3-yl]pyrimidin-5-yl}carbamate of the formula (I), in particular to the modification I, to processes for their preparation, to medicaments comprising them and to their use for fighting diseases.

The invention relates to novel polymorphic forms of methyl {4,6-diamino-2-[1-(2-fluorobenzyl)-1H-pyrazolo[3,4-b]pyridin-3-yl]pyrimidin-5-yl}carbamate of the formula (I), in particular to the modification I, to processes for their preparation, to medicaments comprising them and to their use for fighting diseases.

Methyl {4,6-diamino-2-[1-(2-fluorobenzyl)-1H-pyrazolo[3,4-b]pyridin-3-yl]pyrimidin-5-yl}-carbamate is described in WO 03/095451 and corresponds to the compound of the formula (I):

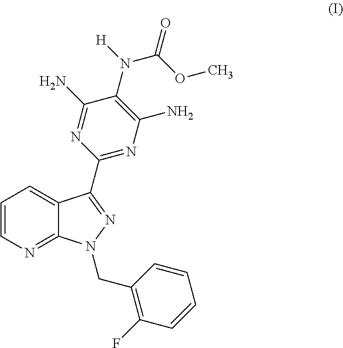

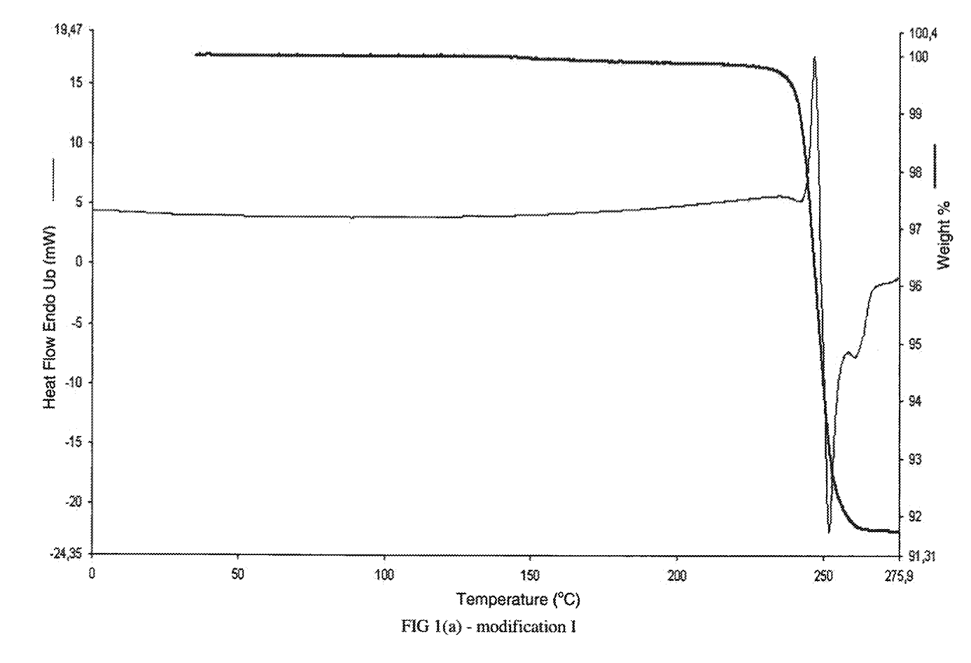

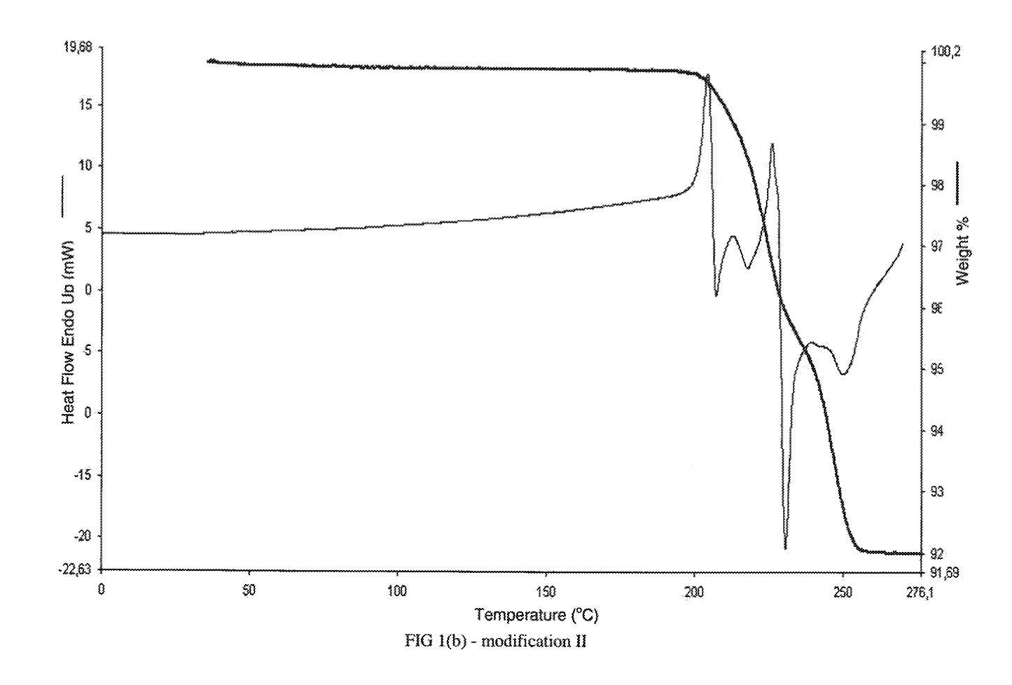

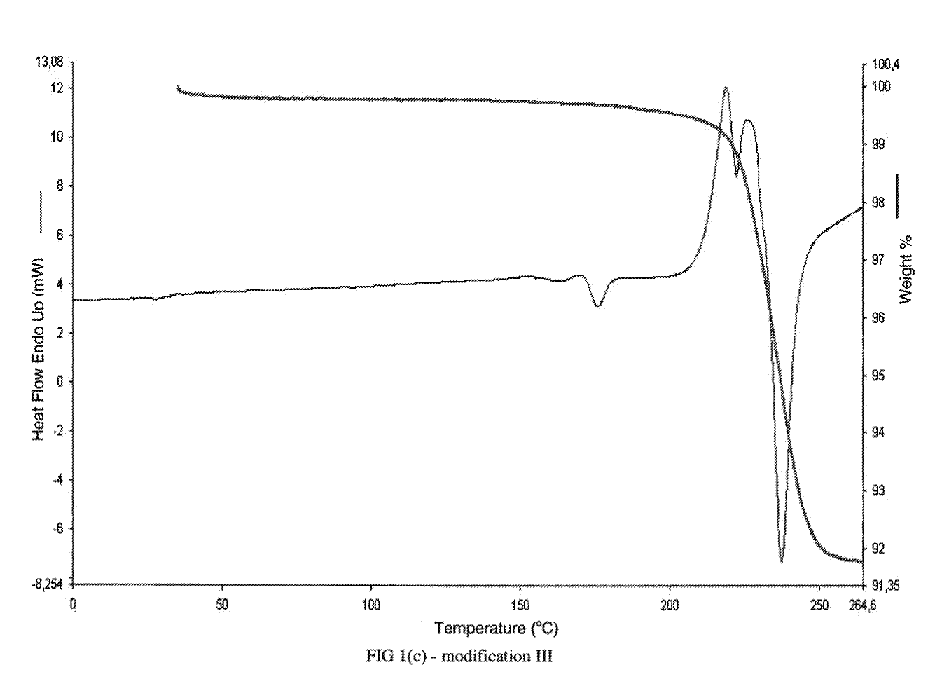

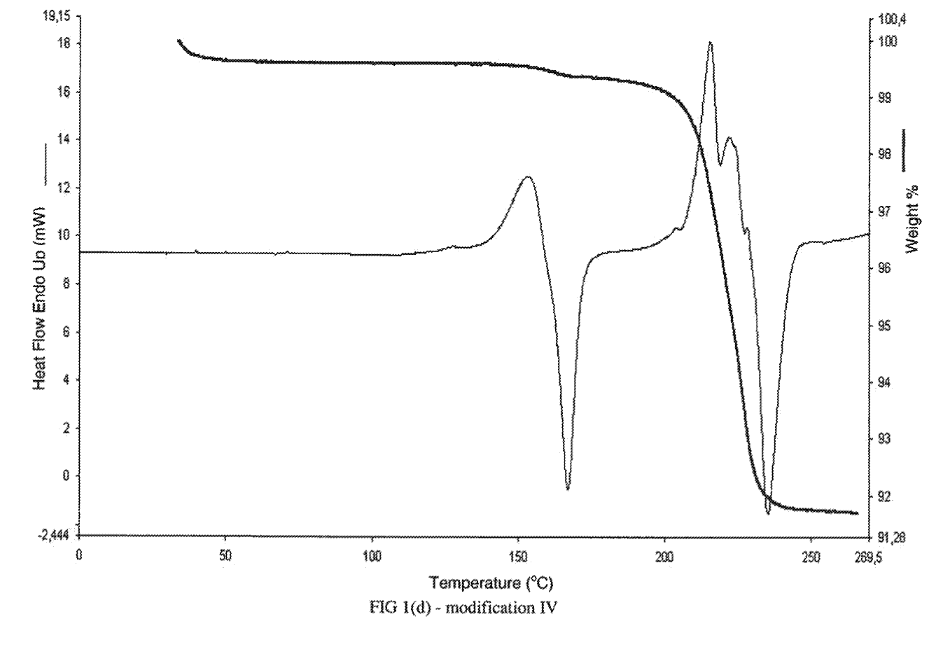

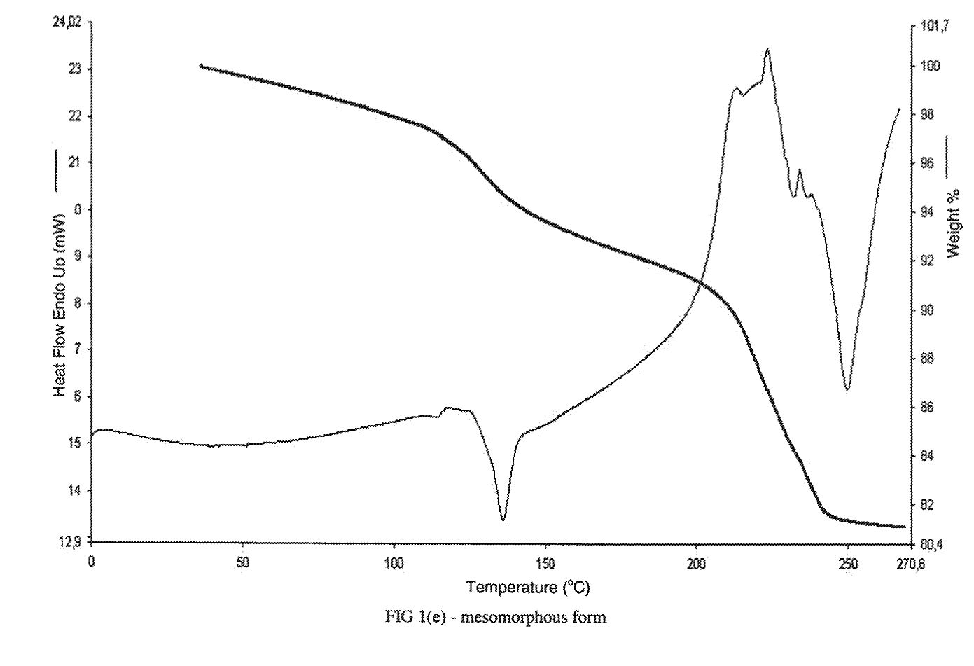

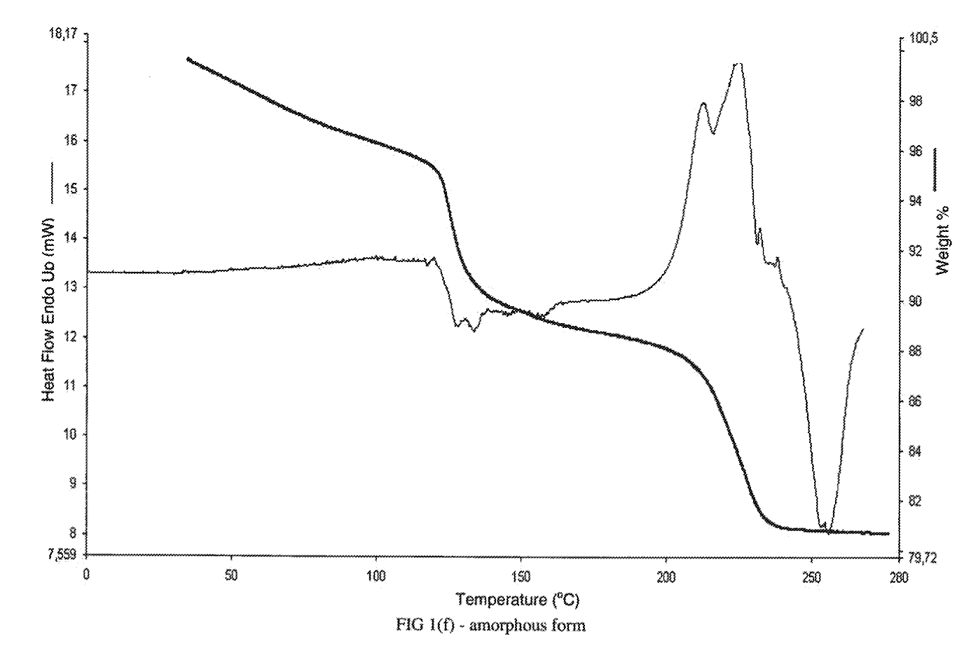

Preparation and use of the compound of the formula (I) for treating, for example, cardiovascular disorders and erectile dysfunction are already known from WO 03/095451. Using the procedure described therein, the compound of the formula (I) is obtained in the form of a crystal modification which is referred to as mesomorphous form hereinbelow. The mesomorphous form has no characteristic melting point. It has a characteristic X-ray diffractogram, IR spectrum, Raman spectrum, FIR spectrum, NIR spectrum and ¹³C solid-state NMR spectrum (Tab. 1-7, FIG. 1-7).

It has now been found that the mesomorphous form is metastable and thus not suitable for use in pharmaceutical formulations such as, for example, solid and semi-solid preparations.

Surprisingly, four further polymorphic forms and the amorphous form have been found. Compared to the mesomorphous form, known from WO 03/095451, the polymorphic forms have markedly different melting points of 244° C. (modification I), 201° C. (modification II), 165° C. (modification III) and 141° C. (modification IV), and each of these modifications has a characteristic X-ray diffractogram, IR spectrum, Raman spectrum, FIR spectrum, NIR spectrum and ¹³C solid-state NMR spectrum (Tab. 1-7, FIG. 1-7).

The present invention provides the compound of the formula (I) in modification I.

The invention provides the compound of the formula (I) in modification I which, in the X-ray diffractogram, has essentially the following preferred peak maximum of the 2 theta angle at 6.1.

The invention preferably provides the compound of the formula (I) in modification I which, in the X-ray diffractogram, has essentially the following preferred peak maximum of the 2 theta angle at 6.1, 14.7 and 22.2.

The invention provides the compound of the formula (I) in modification I which, in the IR spectrum, has essentially the following preferred peak maximum at 3451 cm⁻¹.

The present invention provides the compound of the formula (I) in modification I which, in the NIR spectrum, has essentially the following preferred peak maximum at 6834 cm⁻¹.

General aspects in connection with the present invention are pharmacological properties, processability, preparation process, side-effect profile, stability and pharmacological activity of modification I of the compound of the formula (I).

Surprisingly, the modification I of the compound of the formula (I) is thermodynamically stable and storage-stable even after processing to suspensions. It is therefore suitable in particular for use in pharmaceutical formulations such as, for example, suspensions or cremes, but also in other preparations prepared via suspended active compound, such as, for example, during aqueous granulation or wet grinding. By using, according to the invention, the stable modification I, it is ensured that there are no changes in solubility as a result of a conversion. This increases the safety of preparations comprising the compound of the formula (I), and patient risk is reduced.

In pharmaceutical formulations, the compound of the formula (I) in modification I according to the invention is employed in high purity. For reasons of stability, a pharmaceutical formulation comprises mainly the compound of the formula (I) in modification I and no major amounts of any other form of the compound of the formula (I). Preferably, the medicament comprises more than 90 percent by weight, particularly preferably more than 95 percent by weight, of the compound of the formula (I) in the modification I based on the total amount of the compound of the formula (I) present.

The present invention furthermore provides the use of the compound of the formula (I) in modification I for preparing a medicament for treating diseases, in particular for treating cardiovascular disorders.

The compound of the formula (I) in modification I brings about vasorelaxation and an inhibition of platelet aggregation and leads to a lowering of blood pressure and an increase in the coronary blood flow. These effects are mediated by direct stimulation of soluble guanylate cyclase and an intracellular increase in cGMP.

It can therefore be employed in medicaments for the treatment of cardiovascular disorders such as, for example, for the treatment of high blood pressure and heart failure, stable and unstable Angina pectoris, peripheral and cardiac vascular disorders, of arrhythmias, for the treatment of thromboembolic disorders and ischemias such as myocardial infarction, stroke, transistory and ischemic attacks, disturbances of peripheral blood flow, prevention of restenoses such as after thrombolysis therapies, percutaneous transluminal angioplasties (PTAs), percutaneous transluminal coronary angioplasties (PTCAs), bypass and for the treatment of arteriosclerosis, fibrotic disorders, such as fibrosis of the liver or pulmonary fibrosis, asthmatic disorders and diseases of the urogenital systems such as, for example, prostate hypertrophy, erectile dysfunction, female sexual dysfunction and incontinence and also for the treatment of glaucoma.

It can also be used for fighting diseases of the central nervous system characterized by disturbances of the NO/cGMP system. It is suitable in particular for removing cognitive deficits, for improving learning and memory performances and for treating Alzheimer's disease. It is also suitable for treating disorders of the central nervous system such as states of anxiety, tension and depression, CNS-related sexual dysfunctions and sleep disturbances, and for controlling pathological disturbances of the intake of food, stimulants and addictive substances.

It is furthermore also suitable for regulating cerebral blood flow and thus represents an effective agent for controlling migraine.

It is also suitable for the prophylaxis and control of the sequelae of cerebral infarction (apoplexia cerebri) such as stroke, cerebral ischemias and craniocerebral trauma. It can likewise be employed for controlling states of pain.

In addition, it has an anti-inflammatory effect and can therefore be employed as an anti-inflammatory agent.

Moreover, it is suitable for treating pulmonary arterial hypertension, impaired microcirculation, respiratory infections, reperfusion damage, respiratory disorders, pulmonary disorders and Raynaud's syndrome.

The present invention furthermore provides a method for treating disorders, in particular the disorders mentioned above, using an effective amount of the compound of the formula (I) in modification I.

The compound of the formula (I) in modification I can be administered in a suitable manner, such as, for example, orally, parenterally, pulmonarily, nasally, sublingually, lingually, buccally, rectally, dermally, transdermally, conjunctivally, otically, vaginally, as stents or as an implant.

For these administration routes, the compound according to the invention can be administered in suitable administration forms.

Suitable for oral administration are administration forms working according to the prior art, which release the compound of the formula (I) in modification I rapidly and/or in modified form, such as, for example, tablets (non-coated or coated tablets, for example coated with enteric, slowly dissolving or insoluble coats which control the release of the compound according to the invention), tablets which decompose rapidly in the oral cavity or films/wafers, films/lyophylisates, capsules (for example hard gelatin capsules or soft gelatin capsules), sugar-coated tablets, granules, pellets, powders, suspensions or aerosols.

Parenteral administration can take place with circumvention of an absorption step (for example intravenous, intraarterial, intracardiac, intraspinal or intralumbar) or with involvement of an absorption (for example intramuscular, subcutaneous, intracutaneous, percutaneous or intraperitoneal). For parenteral administration, suitable administration forms are, inter alia, injection and infusion preparations in the form of suspensions, lyophilizates or sterile powders.

Suitable for the other administration routes are, for example, pharmaceutical forms for inhalation (inter alia powder inhalers, nebulizers), tablets, films/wafers or capsules to be applied lingually, sublingually or buccally, suppositories, ear or eye preparations, vaginal capsules, aqueous suspensions (lotions, shake lotions), lipophilic suspensions, ointments, creams, transdermal therapeutic systems (such as, for example, patches), pastes, dusting powders, implants or stents.

The compound according to the invention can be converted into the administration forms mentioned. This may take place in a manner known per se by mixing with inert non-toxic, pharmaceutically acceptable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants such as, for example, ascorbic acid), colours (e.g. inorganic pigments such as, for example, iron oxides) and taste and/or odour corrigents.

The present invention furthermore provides medicaments comprising at least the compound of the formula (I) in modification I, usually together with one or more inert non-toxic, pharmaceutically suitable auxiliaries such as, for example, binders, fillers, etc., and their use for the purposes mentioned above.

In general, it has been found to be advantageous to administer the compound according to the invention in total amounts of from about 0.5 to about 500, preferably from 5 to 100, mg/kg of body weight per day, if appropriate in the form of a plurality of individual doses, to obtain the desired results. An individual dose contains the active compound in amounts of from about 1 to about 80, preferably 3 to 30, mg/kg of body weight.

The invention furthermore provides a process for preparing the compound of the formula (I) in modification I, by suspending the compound of the formula (I) for example in the mesomorphous form, in an inert solvent and stirring or shaking at a temperature of from 10° C. to the reflux temperature of the solvent, preferably at from 15° C. to 35° C., particularly preferably at from 20 to 30° C., until the desired degree of conversion has been achieved, particularly preferably to quantitative conversion into modification I. The resulting crystals of modification I are separated off and, to remove the solvent present, dried at room temperature or at elevated temperature until the weight remains constant.

Suitable inert solvents are lower alcohols, such as methanol, ethanol, n-propanol, isopropanol, n-butanol, sec-butanol, iso-butanol, 1-pentanol, or ketones, such as acetone, or alkanes, such as n-pentane, cyclopentane, n-hexane, cyclohexane, or tetrahydrofuran, acetonitrile, toluene, ethyl acetate, 1,4-dioxane or mixtures of the solvents mentioned. Preference is given to acetonitrile and acetone or mixtures of the solvents mentioned.

In general, the preparation processes are carried out under atmospheric pressure. However, it is also possible to operate under elevated or reduced pressure, for example at from 0.5 to 5 bar.

The percentages in the tests and examples below are, unless indicated otherwise, percentages by weight; parts are parts by weight. Solvent ratios, dilution ratios and concentrations of liquid/liquid solutions are in each case by volume.

### EXPERIMENTAL PART

### Working Examples

The DSC thermograms were recorded using a Differential Scanning calorimeter DSC 7, Pyris-1 or Diamond from Perkin-Elmer using a heating rate of 20 Kmin⁻¹ The measurements were carried out in perforated aluminium crucibles, the purge gas used was nitrogen. There was no sample preparation.

The TGA measurements were carried out using TGA7 and Pyris-1-TGA thermobalances from Perkin-Elmer using a heating rate of 10 Kmin⁻¹. The measurements were carried out in open platinum crucibles, the purge gas used was nitrogen. There was no sample preparation.

The X-ray diffractograms were recorded using an STOE STADI-P transmission diffractometer having a position-sensitive detector (PSD2) at room temperature (radiation: copper, Kα1, primary monochromator: Ge [1 1 1], wavelength:1.5406 Å).

The Raman spectra were recorded using RFS 100 and Multi RAM FT-Raman spectrometers from Bruker at room temperature. The resolution was 2 cm⁻¹. There was no sample preparation. The measurement was carried out in glass tubes or on an aluminium disc.

The IR spectra were recorded using Vertex 80v and IFS 66v FT-IR spectrometers from Bruker at room temperature. The resolution was 2 cm⁻¹. The measurement was carried out in a KBr matrix as pressed disc.

The FIR spectra were recorded using Vertex 80v and IFS 66v FT-IR spectrometers from Bruker at room temperature. The resolution was 2 cm⁻¹. The measurement was carried out in a polyethylene matrix as pressed disc.

The NIR spectra were recorded using an IFS 28/N FT-NIR spectrometer from Bruker at room temperature. The resolution was 8 cm⁻¹. There was no sample preparation.

The solid-state ¹³C-NMR spectra were recorded using a DRX 400 spectrometer from Bruker at room temperature. The measuring frequency was 100.6 MHz and the rotation frequencies were 8500 Hz and 10000 Hz. There was no sample preparation.

### Example 1

### Preparation of methyl {4,6-diamino-2-[1-(2-fluorobenzyl)-1H-pyrazolo[3,4-b]pyridin-3-yl]pyrimidin-5-yl}-carbamate of the formula (I) in modification I

### Example 1.1

About 100 mg of methyl {4,6-diamino-2-[1-(2-fluorobenzyl)-1H-pyrazolo[3,4-b]pyridin-3-yl]pyrimidin-5-yl}carbamate of the formula (I) in the mesomorphous form are suspended in 3 ml of acetonitrile and stirred at room temperature. After one week, the suspension is filtered and the residue is dried at room temperature and atmospheric humidity. The residue is examined by X-ray diffractometry and corresponds to the title compound in modification I.

### Example 1.2

About 100 mg of methyl {4,6-diamino-2-[1-(2-fluorobenzyl)-1H-pyrazolo[3,4-b]pyridin-3-yl]pyrimidin-5-yl}carbamate of the formula (I) in the mesomorphous form are suspended in 2 ml of acetone and stirred at 50° C. under reflux. After one week, the suspension is filtered and the residue is dried at room temperature and atmospheric humidity. The residue is examined by X-ray diffractometry and corresponds to the title compound in modification I.

### Example 1.3

7.1 kg of methyl {4,6-diamino-2-[1-(2-fluorobenzyl)-1H-pyrazolo[3,4-b]pyridin-3-yl]pyrimidin-5-yl}carbamate of the formula (I) as di-DMSO solvate are suspended in 171.6 kg of ethyl acetate and 42 kg of ethanol and stirred at about 73° C. under reflux for 20 h. The suspension is cooled to RT and filtered off with suction, and the filter cake is washed with ethyl acetate and water. The moist product is dried at 50° C. under reduced pressure. The product is examined by X-ray diffractometry and corresponds to the title compound in modification I.

### Example 2

### Preparation of methyl {4,6-diamino-2-[1-(2-fluorobenzyl)-1H-pyrazolo[3,4-b]pyridin-3-yl]pyrimidin-5-yl}carbamate of the formula (I) in modification II

### Example 2.1

110.5 g of methyl {4,6-diamino-2-[1-(2-fluorobenzyl)-1H-pyrazolo[3,4-b]pyridin-3-yl]pyrimidin-5-yl}carbamate of the formula (I) as HCl salt are suspended in 1960 ml of ethanol at room temperature. 140 ml of triethylamine are metered in, and the mixture is stirred at RT for 3 h. The solid is filtered off with suction and washed with ethanol. The moist product is dried at 50° C. under reduced pressure overnight. The product is examined by X-ray diffractometry and corresponds to the title compound in modification II.

### Example 3

### Preparation of methyl {4,6-diamino-2-[1-(2-fluorobenzyl)-1H-pyrazolo[3,4-b]pyridin-3-yl]pyrimidin-5-yl}carbamate of the formula (I) in modification III

### Example 3.1

3.1 g of methyl {4,6-diamino-2-[1-(2-fluorobenzyl)-1H-pyrazolo[3,4-b]pyridin-3-yl]pyrimidin-5-yl}carbamate of the formula (I) in modification II are suspended in 60 ml of methanol and stirred at RT. After one week, the suspension is filtered and the residue is dried at room temperature and atmospheric humidity. The active compound is then heat-conditioned at 125° C. for 20 min. The active compound is examined by X-ray diffractometry and corresponds to the title compound in modification III.

### Example 4

### Preparation of methyl {4,6-diamino-2-[1-(2-fluorobenzyl)-1H-pyrazolo[3,4-b]pyridin-3-yl]pyrimidin-5-yl}carbamate of the formula (I) in modification IV

### Example 4.1

3 g of methyl {4,6-diamino-2-[1-(2-fluorobenzyl)-1H-pyrazolo[3,4-b]pyridin-3-yl]pyrimidin-5-yl}carbamate of the formula (I) in modification II are suspended in 60 ml of acetone and stirred at −20° C. After two weeks, the suspension is filtered and the residue is dried at room temperature and atmospheric humidity. The active compound is then heat-conditioned at 125° C. for 30 min. The active compound is examined by X-ray diffractometry and corresponds to the title compound in modification IV.

### Example 5

### Preparation of the amorphous form of methyl {4,6-diamino-2-[1-(2-fluorobenzyl)-1H-pyrazolo[3,4-b]pyridin-3-yl]pyrimidin-5-yl}carbamate of the formula (I)

### Example 5.1

3 g of methyl {4,6-diamino-2-[1-(2-fluorobenzyl)-1H-pyrazolo[3,4-b]pyridin-3-yl]pyrimidin-5-yl}carbamate of the formula (I) in modification I are dissolved in 1.1 1 of hot tetrahydrofuran, and the solution is filtered and allowed to stand at room temperature at atmospheric humidity until the solvent has evaporated. The residue is examined by X-ray diffractometry and corresponds to the title compound in the amorphous form.

TABLE 1
Differential scanning calorimetry and thermogravimetry

| | Melting point | |
|---|---|---|
| | (decomposition) | Mass loss |
| | [° C.] | [% by weight] |
| modification I | 244 | <0.5 |
| modification II | 201 | <0.5 |
| modification III | 165 | <0.5 |
| modification IV | 141 | <1 |
| mesomorphous | — | about 8 |
| amorphous | — | about 10 |

TABLE 2
X-ray diffractometry
Peak maximum [2 theta]

| Modifica- | Modifica- | Modifica- | Modifica- | meso- |
|---|---|---|---|---|
| tion I | tion II | tion III | tion IV | morphous |
| 3.6 | 8.3 | 6.6 | 10.6 | 4.0 |
| 4.9 | 11.3 | 7.7 | 11.6 | 5.2 |
| 6.1 | 11.5 | 13.9 | 12.8 | 6.1 |
| 7.0 | 12.1 | 15.1 | 13.4 | 9.1 |
| 7.3 | 13.6 | 15.6 | 13.8 | 13.0 |
| 8.8 | 14.1 | 16.8 | 14.1 | 15.2 |
| 9.9 | 14.8 | 17.4 | 16.1 | 16.9 |
| 10.9 | 16.3 | 17.5 | 16.5 | 17.5 |
| 12.0 | 17.0 | 17.8 | 17.6 | 21.0 |
| 12.3 | 17.5 | 18.2 | 17.8 | 23.7 |
| 14.7 | 18.2 | 19.5 | 18.0 | 25.6 |
| 15.3 | 19.0 | 19.8 | 18.6 | |
| 16.5 | 21.1 | 20.5 | 19.0 | |
| 17.6 | 22.1 | 22.7 | 19.6 | |
| 18.2 | 22.9 | 23.0 | 20.1 | |
| 18.4 | 23.3 | 23.3 | 21.0 | |
| 19.8 | 24.0 | 23.7 | 21.4 | |
| 20.8 | 25.1 | 24.1 | 21.9 | |
| 21.1 | 25.4 | 24.4 | 22.8 | |
| 21.3 | 26.1 | 25.4 | 24.5 | |
| 21.8 | 26.7 | 26.3 | 25.3 | |
| 22.2 | 28.6 | 26.8 | 25.4 | |
| 22.9 | 29.3 | 28.1 | 26.2 | |
| 24.1 | 30.4 | 28.7 | 26.5 | |
| 24.4 | 34.0 | 30.4 | 27.5 | |
| 24.7 | 35.6 | 31.2 | 28.1 | |
| 25.6 | 36.9 | 32.8 | 28.3 | |
| 26.0 | 37.7 | 33.8 | 28.6 | |
| 26.8 | | 35.1 | 30.5 | |
| 27.4 | | 37.6 | 32.5 | |
| 27.8 | | | 33.5 | |
| 28.1 | | | 33.8 | |
| 28.3 | | | 35.6 | |
| 29.3 | | | 36.2 | |
| 29.7 | | | | |
| 30.1 | | | | |
| 30.9 | | | | |
| 31.7 | | | | |
| 32.0 | | | | |
| 32.7 | | | | |
| 33.0 | | | | |
| 33.5 | | | | |
| 34.2 | | | | |
| 35.3 | | | | |
| 35.6 | | | | |
| 36.0 | | | | |

TABLE 3
IR spectroscopy
Wave number [cm⁻¹]

| Modifica- | Modifica- | Modifica- | Modifica- | meso- | |
|---|---|---|---|---|---|
| tion I | tion II | tion III | tion IV | morphous | amorphous |
| 3483 | 3507 | 3503 | 3489 | 3633 | 3451 |
| 3470 | 3484 | 3409 | 3287 | 3443 | 3331 |
| 3451 | 3397 | 3365 | 3157 | 3330 | 3217 |
| 3387 | 3291 | 3268 | 2954 | 3222 | 3150 |
| 3330 | 3158 | 3092 | 1710 | 2952 | 2953 |
| 3276 | 3024 | 3022 | 1628 | 1705 | 1707 |
| 3214 | 2955 | 2987 | 1561 | 1630 | 1628 |
| 3133 | 1724 | 2949 | 1515 | 1566 | 1566 |
| 2952 | 1632 | 2843 | 1492 | 1511 | 1510 |
| 1712 | 1608 | 1733 | 1480 | 1492 | 1492 |
| 1636 | 1562 | 1627 | 1439 | 1477 | 1478 |
| 1567 | 1491 | 1609 | 1363 | 1437 | 1438 |
| 1509 | 1477 | 1563 | 1342 | 1390 | 1390 |
| 1478 | 1437 | 1511 | 1324 | 1351 | 1351 |
| 1441 | 1386 | 1492 | 1302 | 1323 | 1323 |
| 1387 | 1345 | 1477 | 1288 | 1288 | 1289 |
| 1350 | 1322 | 1454 | 1247 | 1277 | 1276 |
| 1323 | 1287 | 1438 | 1187 | 1247 | 1248 |
| 1289 | 1275 | 1388 | 1169 | 1232 | 1233 |
| 1276 | 1235 | 1356 | 1144 | 1174 | 1175 |
| 1249 | 1170 | 1322 | 1112 | 1140 | 1141 |
| 1232 | 1141 | 1288 | 1090 | 1112 | 1112 |
| 1174 | 1112 | 1274 | 1075 | 1061 | 1061 |
| 1139 | 1087 | 1250 | 1059 | 1030 | 1031 |
| 1111 | 1071 | 1230 | 1031 | 940 | 940 |
| 1086 | 1030 | 1185 | 939 | 911 | 911 |
| 1075 | 995 | 1173 | 910 | 863 | 864 |
| 1062 | 937 | 1140 | 871 | 846 | 847 |
| 1031 | 907 | 1109 | 857 | 820 | 820 |
| 1004 | 874 | 1095 | 849 | 808 | 808 |
| 941 | 849 | 1068 | 822 | 797 | 796 |
| 911 | 812 | 1034 | 807 | 774 | 774 |
| 867 | 799 | 974 | 799 | 757 | 758 |
| 849 | 781 | 940 | 777 | 712 | 713 |
| 820 | 665 | 911 | 765 | 643 | 621 |
| 808 | 671 | 865 | 753 | 621 | 591 |
| 795 | 644 | 839 | 708 | 590 | 576 |
| 774 | 627 | 820 | 683 | 568 | 534 |
| 760 | 587 | 806 | 642 | 534 | 519 |
| 715 | 570 | 795 | 593 | 519 | |
| 631 | 536 | 777 | 571 | | |
| 594 | | 762 | 531 | | |
| 576 | | 704 | | | |
| 535 | | 641 | | | |
| | | 87 | | | |
| | | 572 | | | |
| | | 533 | | | |
| | | 513 | | | |

TABLE 4
Raman spectroscopy
Wave number [cm⁻¹]

| Modifica- | Modifica- | Modifica- | Modifica- | meso- | |
|---|---|---|---|---|---|
| tion I | tion II | tion III | tion IV | morphous | amorphous |
| 3452 | 3397 | 3081 | 3068 | 3067 | 3332 |
| 3387 | 3143 | 3023 | 3028 | 3024 | 3067 |
| 3331 | 3095 | 2985 | 2942 | 2956 | 3030 |
| 3086 | 3079 | 2947 | 2850 | 2608 | 2955 |
| 3054 | 3056 | 2929 | 1704 | 1704 | 2843 |
| 3022 | 3025 | 2844 | 1621 | 1618 | 2592 |
| 2990 | 3006 | 2589 | 1599 | 1578 | 2329 |
| 2953 | 2956 | 1730 | 1576 | 1508 | 1703 |
| 2834 | 2843 | 1633 | 1507 | 1479 | 1617 |
| 2604 | 1728 | 1616 | 1486 | 1448 | 1577 |
| 1702 | 1641 | 1597 | 1444 | 1423 | 1508 |
| 1633 | 1619 | 1565 | 1421 | 1380 | 1479 |
| 1618 | 1567 | 1504 | 1389 | 1323 | 1447 |
| 1598 | 1503 | 1483 | 1373 | 1309 | 1423 |
| 1577 | 1478 | 1440 | 1343 | 1278 | 1380 |
| 1508 | 1442 | 1421 | 1323 | 1252 | 1354 |
| 1477 | 1434 | 1385 | 1303 | 1233 | 1323 |
| 1447 | 1417 | 1367 | 1251 | 1177 | 1309 |
| 1420 | 1385 | 1329 | 1232 | 1157 | 1250 |
| 1380 | 1372 | 1289 | 1170 | 1142 | 1232 |
| 1351 | 1344 | 1250 | 1156 | 1114 | 1176 |
| 1322 | 1322 | 1229 | 1145 | 1063 | 1157 |
| 1307 | 1308 | 1186 | 1115 | 1036 | 1142 |
| 1289 | 1288 | 1174 | 1062 | 964 | 1113 |
| 1277 | 1277 | 1143 | 1035 | 823 | 1062 |
| 1249 | 1244 | 1109 | 1006 | 798 | 1035 |
| 1225 | 1232 | 1061 | 964 | 777 | 963 |
| 1175 | 1172 | 1031 | 824 | 742 | 911 |
| 1157 | 1144 | 996 | 807 | 717 | 823 |
| 1140 | 1115 | 958 | 799 | 645 | 797 |
| 1112 | 1059 | 912 | 773 | 591 | 776 |
| 1064 | 1032 | 820 | 739 | 560 | 742 |
| 1034 | 964 | 805 | 719 | 536 | 716 |
| 961 | 906 | 796 | 647 | 521 | 645 |
| 910 | 820 | 784 | 598 | 472 | 591 |
| 823 | 800 | 776 | 564 | 447 | 566 |
| 808 | 772 | 744 | 535 | 408 | 536 |
| 796 | 740 | 706 | 446 | 368 | 521 |
| 777 | 719 | 558 | 403 | 331 | 471 |
| 773 | 646 | 540 | 350 | 265 | 447 |
| 768 | 629 | 599 | 320 | 221 | 410 |
| 741 | 588 | 589 | 264 | 190 | 368 |
| 717 | 556 | 566 | 247 | 158 | 332 |
| 644 | 538 | 534 | 231 | | 264 |
| 632 | 526 | 514 | 220 | | 220 |
| 592 | 467 | 479 | 194 | | 190 |
| 559 | 436 | 439 | 140 | | 157 |
| 534 | 349 | 403 | | | 117 |
| 465 | 316 | 364 | | | |
| 447 | 268 | 327 | | | |
| 265 | | 287 | | | |
| 233 | | 257 | | | |
| 296 | | 231 | | | |
| 266 | | 219 | | | |
| 246 | | 188 | | | |
| 215 | | 156 | | | |
| 189 | | | | | |
| 160 | | | | | |

TABLE 5
FIR spectroscopy
Wave number [cm⁻¹]

| Modifica- | Modifica- | Modifica- | Modifica- | meso- | |
|---|---|---|---|---|---|
| tion I | tion II | tion III | tion IV | morphous | amorphous |
| 487 | 461 | 478 | 454 | 495 | 496 |
| 466 | 447 | 437 | 425 | 485 | 489 |
| 451 | 435 | 402 | 393 | 469 | 484 |
| 430 | 426 | 363 | 379 | 447 | 481 |
| 407 | 405 | 334 | 368 | 430 | 471 |
| 365 | 362 | 310 | 340 | 406 | 463 |
| 343 | 346 | 284 | 322 | 368 | 447 |
| 325 | 324 | 231 | 246 | 331 | 436 |
| 318 | 304 | 189 | 238 | 289 | 430 |
| 291 | 246 | 168 | 216 | 262 | 407 |
| 262 | 240 | 105 | 196 | 236 | 329 |
| 237 | 212 | | 156 | 188 | 289 |
| 218 | 194 | | 113 | 160 | 263 |
| 188 | 167 | | | 108 | 237 |
| 160 | 146 | | | 92 | 189 |
| 96 | 104 | | | | 158 |
| | 96 | | | | 96 |
| | 92 | | | | |
| | 84 | | | | |

TABLE 6
NIR spectroscopy
Wave number [cm⁻¹]

| Modifica- | Modifica- | Modifica- | Modifica- | meso- | |
|---|---|---|---|---|---|
| tion I | tion II | tion III | tion IV | morphous | amorphous |
| 9793 | 9786 | 9905 | 8809 | 8789 | 8797 |
| 8779 | 8798 | 8845 | 8462 | 8408 | 8420 |
| 7828 | 8534 | 8687 | 7851 | 7107 | 7109 |
| 6834 | 8450 | 8472 | 6857 | 6846 | 6850 |
| 6724 | 8152 | 7852 | 6667 | 6636 | 6637 |
| 6631 | 7866 | 7305 | 6011 | 5977 | 5976 |
| 6328 | 6949 | 5966 | 5106 | 5244 | 5236 |
| 6059 | 6842 | 5875 | 5064 | 5057 | 5057 |
| 5984 | 6784 | 5782 | 4971 | 4984 | 4984 |
| 5846 | 6666 | 5722 | 4795 | 4802 | 4798 |
| 5593 | 6357 | 5430 | 4741 | 4660 | 4660 |
| 5095 | 6044 | 5028 | 4659 | 4432 | 4432 |
| 5058 | 5971 | 5920 | 4538 | 4149 | 4148 |
| 4965 | 5874 | 5846 | 4486 | 4056 | 4053 |
| 4916 | 5811 | 5755 | 4439 | | |
| 4865 | 5625 | 5720 | 4216 | | |
| 4808 | 5429 | 5627 | 4155 | | |
| 4646 | 5231 | 5244 | 4092 | | |
| 4595 | 5107 | 5116 | | | |
| 4531 | 5067 | 5071 | | | |
| 4485 | 5004 | 4010 | | | |
| 4419 | 4965 | 4974 | | | |
| 4348 | 4891 | 4899 | | | |
| 4268 | 4836 | 4763 | | | |
| 4199 | 4805 | 4665 | | | |
| 4062 | 4732 | 4546 | | | |
| | 4659 | 4491 | | | |
| | 4553 | 4433 | | | |
| | 4503 | 4386 | | | |
| | 4481 | 4338 | | | |
| | 4443 | 4224 | | | |
| | 4402 | 4175 | | | |
| | 4367 | 4049 | | | |
| | 4329 | | | | |
| | 4262 | | | | |
| | 4164 | | | | |
| | 4120 | | | | |
| | 4057 | | | | |
| | 4037 | | | | |

TABLE 7
¹³C solid-state NMR spectroscopy
ppm

| Modifica- | Modifica- | Modifica- | Modifica- | meso- | |
|---|---|---|---|---|---|
| tion I | tion II | tion III | tion IV | morphous | amorphous |
| 52 | 53 | 31 | 51 | 22 | 32 |
| 95 | 94 | 44 | 94 | 26 | 35 |
| 116 | 116 | 52 | 117 | 31 | 42 |
| 123 | 122 | 95 | 125 | 35 | 53 |
| 126 | 124 | 116 | 128 | 41 | 96 |
| 128 | 130 | 123 | 134 | 52 | 115 |
| 130 | 131 | 133 | 141 | 96 | 125 |
| 133 | 135 | 142 | 144 | 115 | 133 |
| 138 | 142 | 150 | 147 | 124 | 142 |
| 141 | 147 | 158 | 150 | 128 | 150 |
| 149 | 149 | 161 | 158 | 132 | 159 |
| 150 | 150 | | 161 | 141 | 162 |
| 158 | 154 | | | 149 | |
| 161 | 158 | | | 158 | |
| | 161 | | | 161 | |

FIG. 1: DSC and TGA thermograms of modifications I-IV, the mesomorphous form and the amorphous form

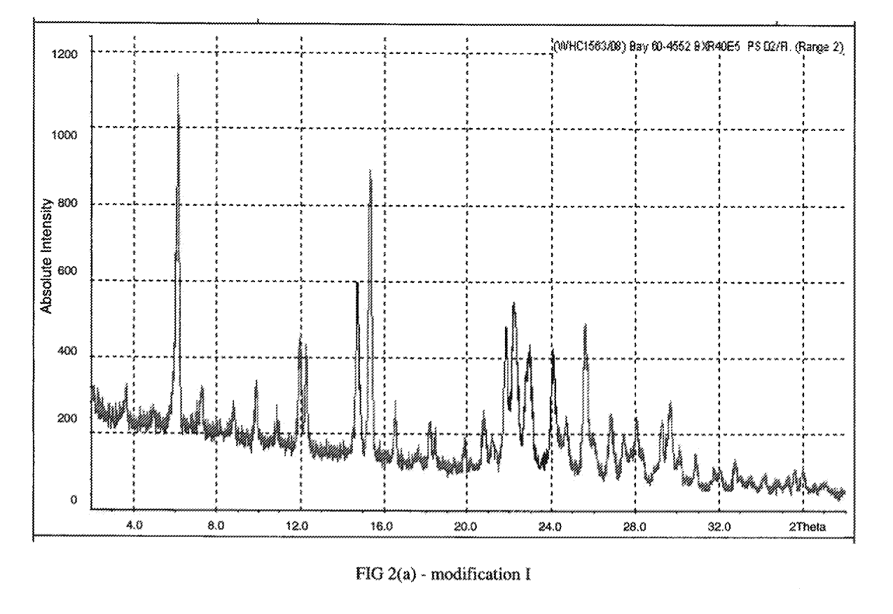

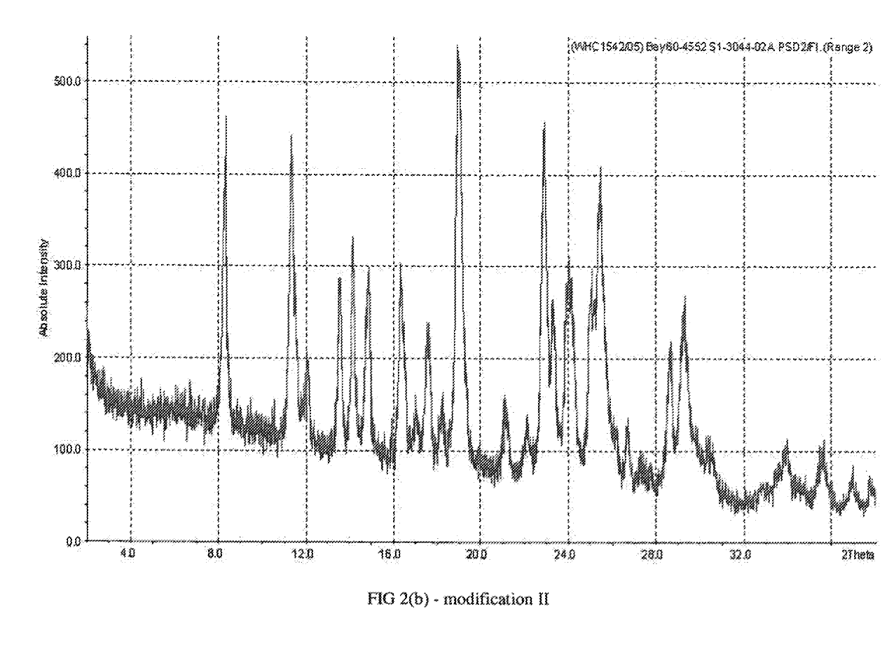

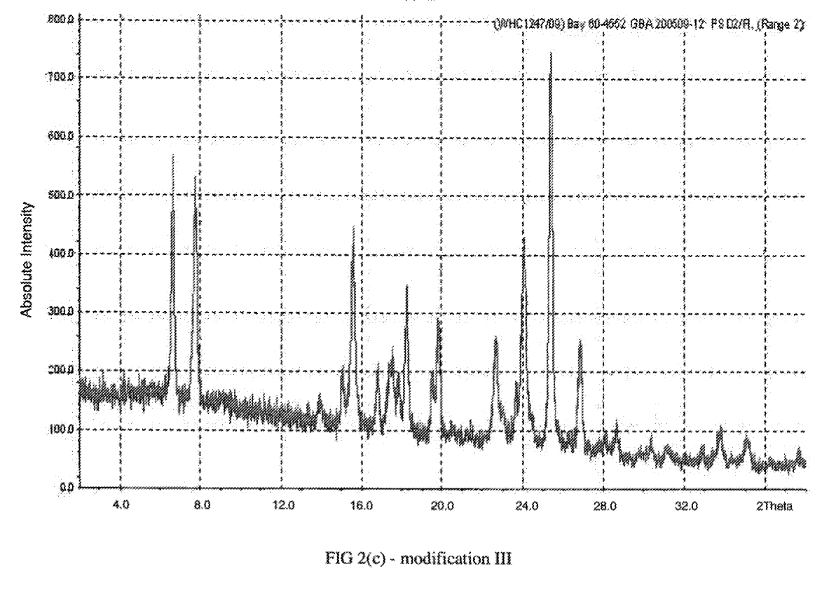

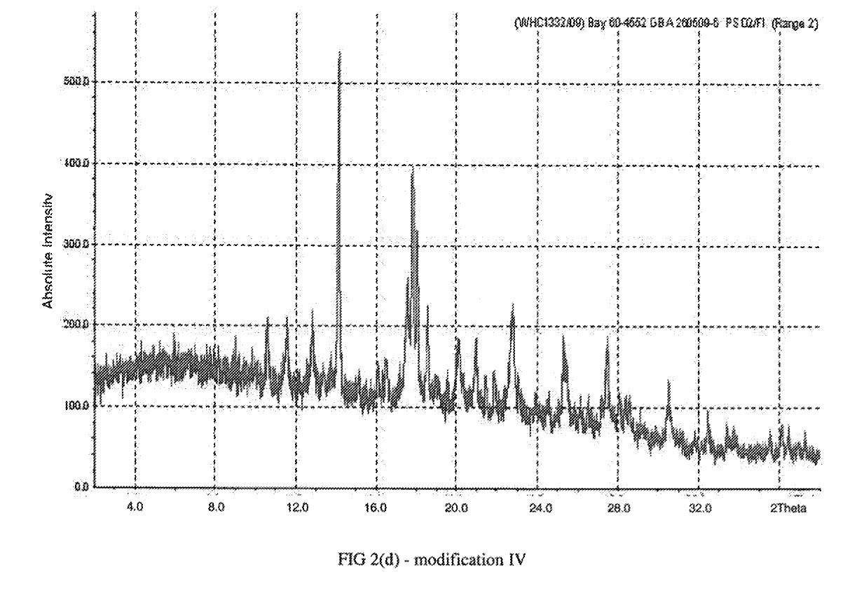

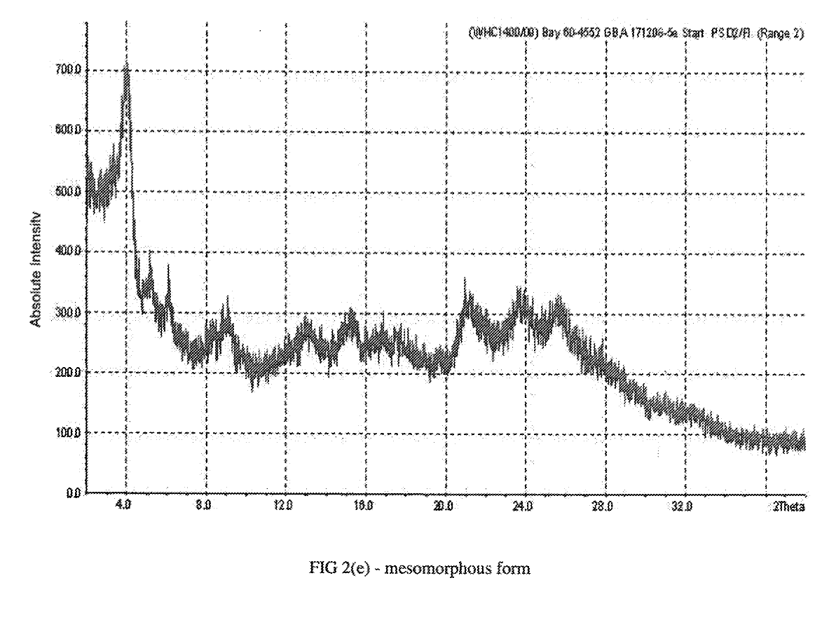

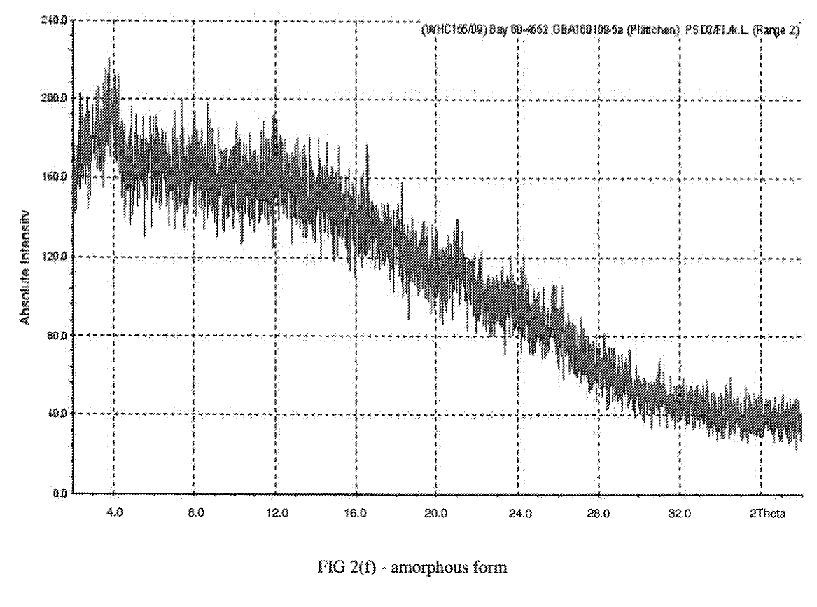

FIG. 2: X-ray diffractograms of modifications I-IV, the mesomorphous form and the amorphous form

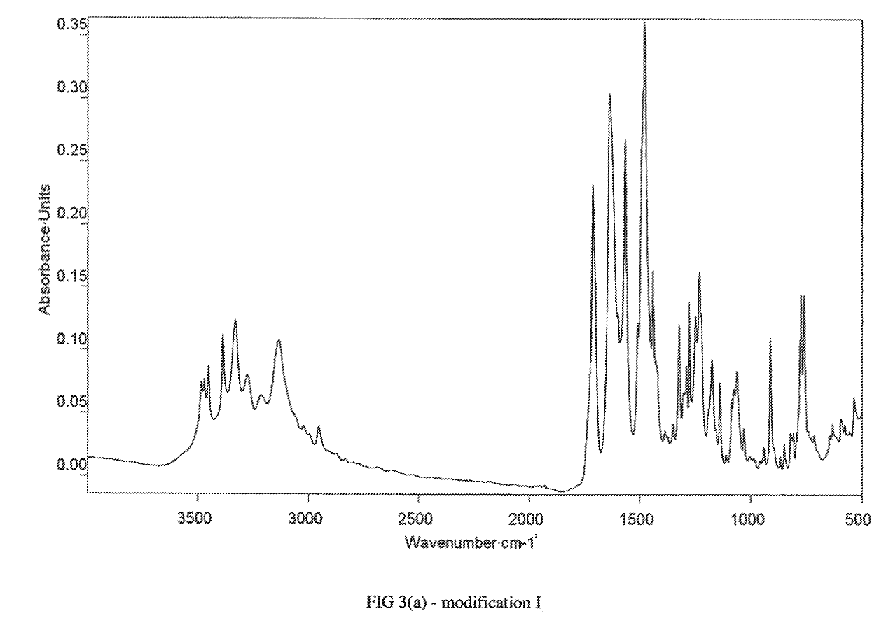

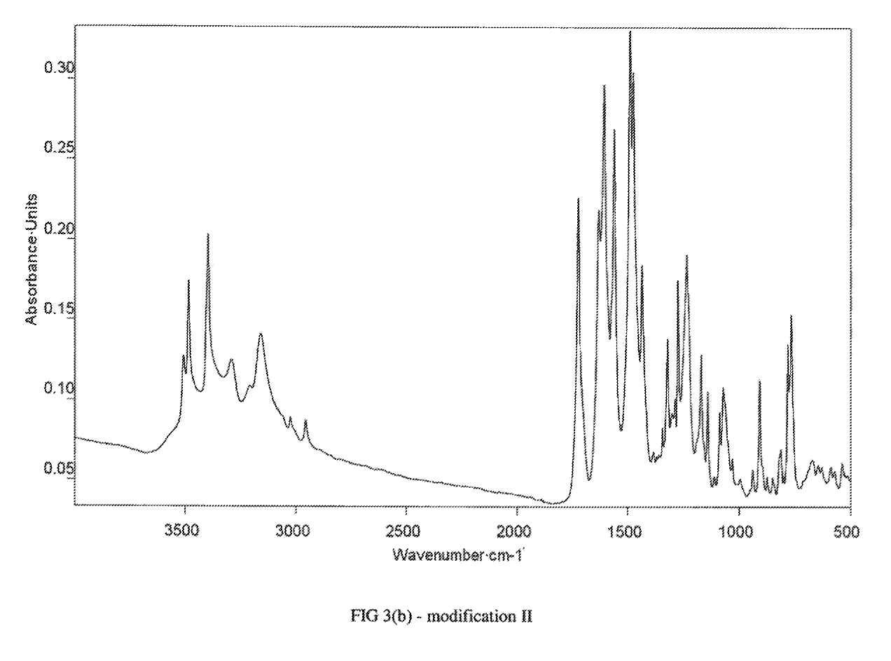

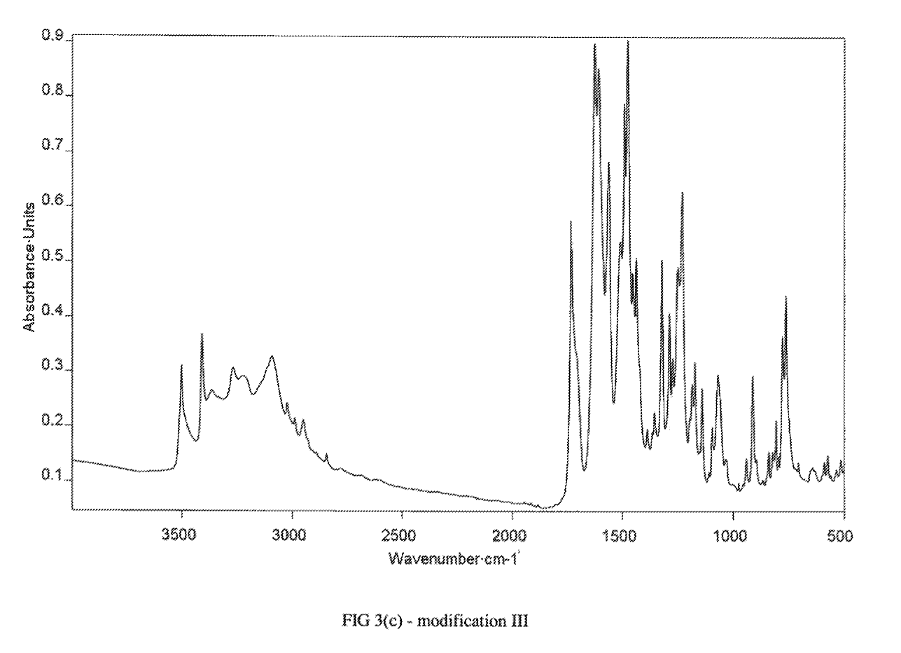

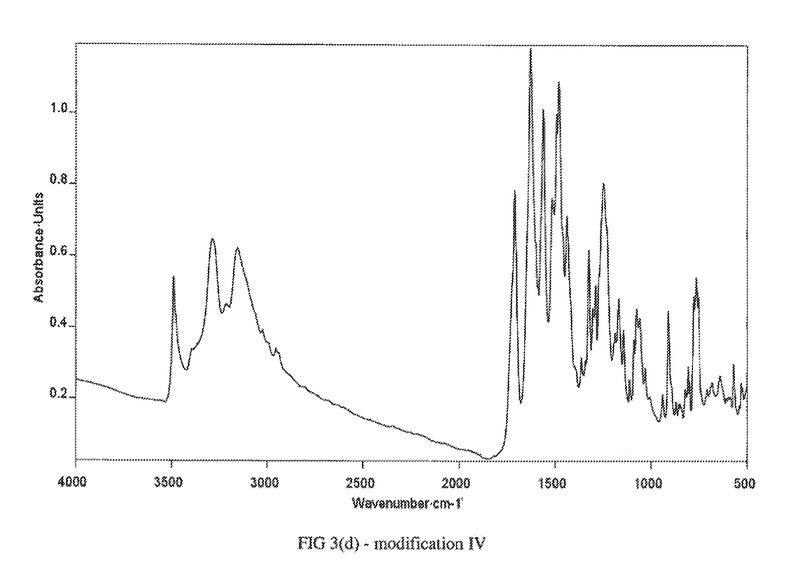

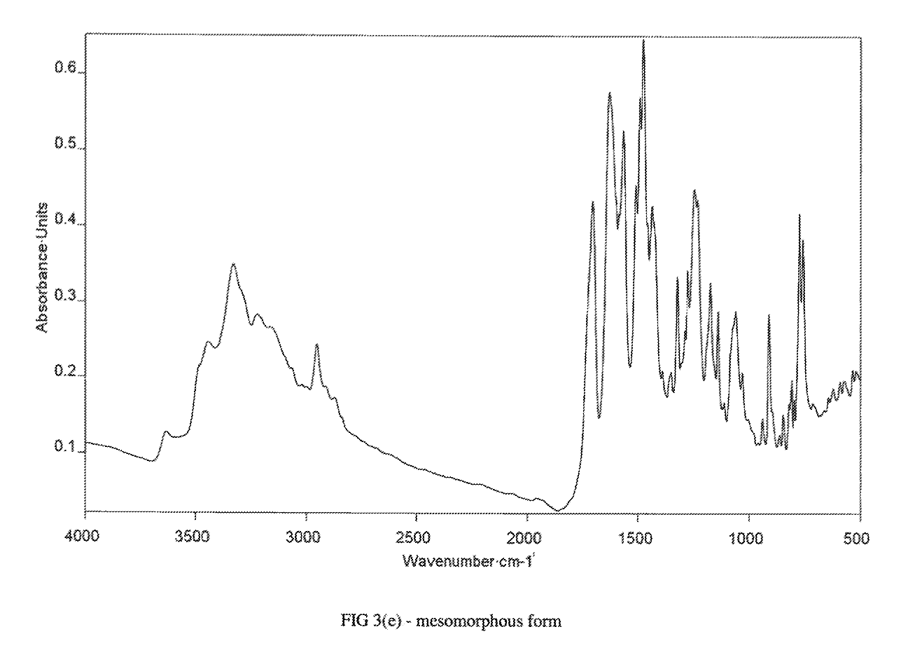

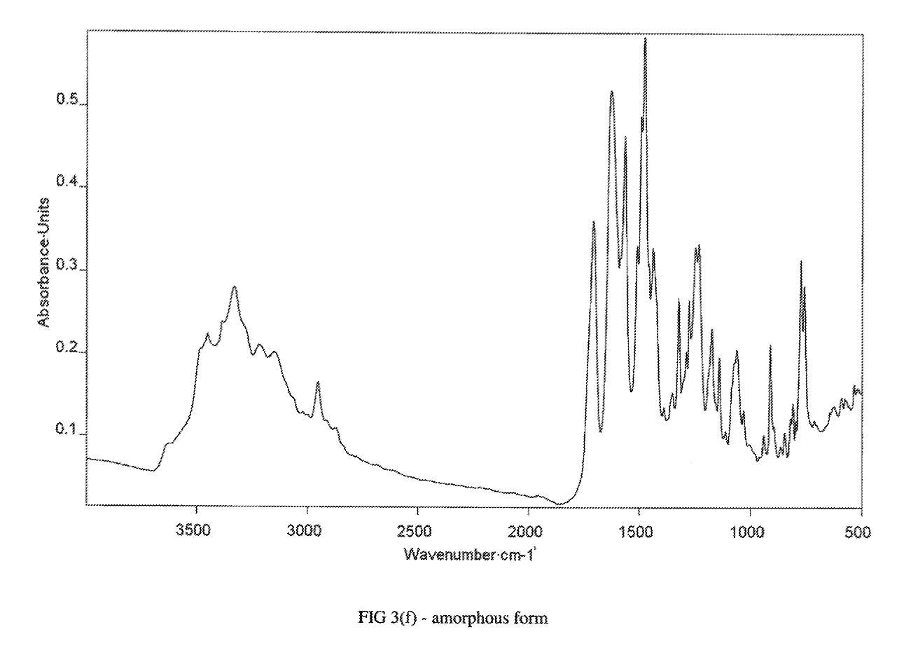

FIG. 3: IR spectra of modifications I-IV, the mesomorphous form and the amorphous form

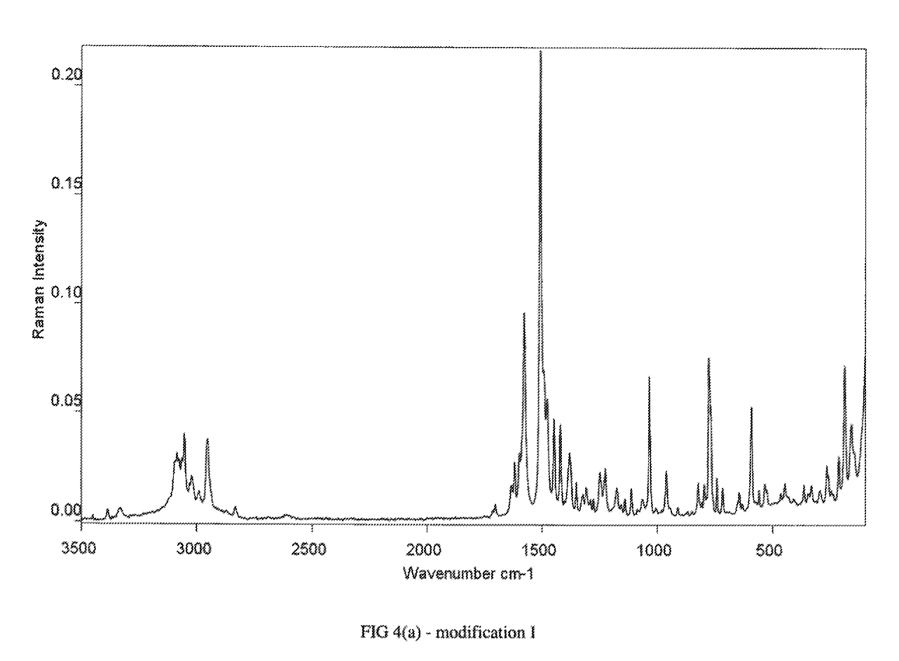

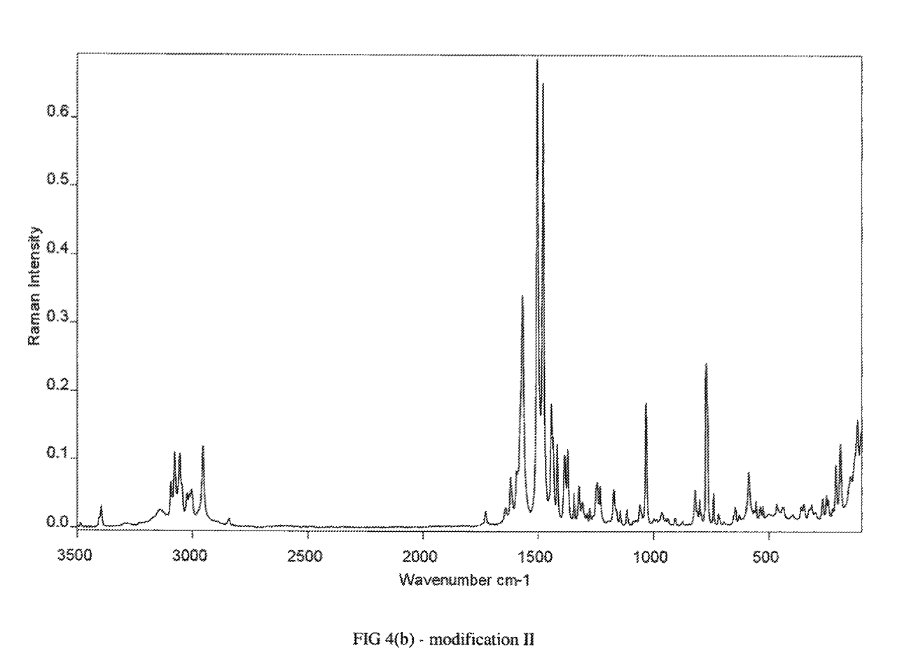

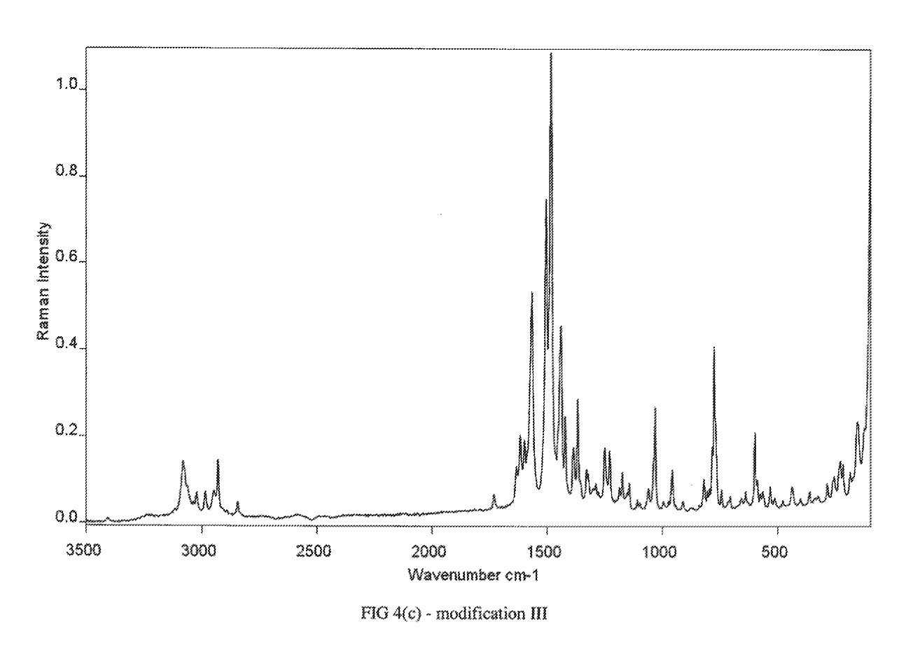

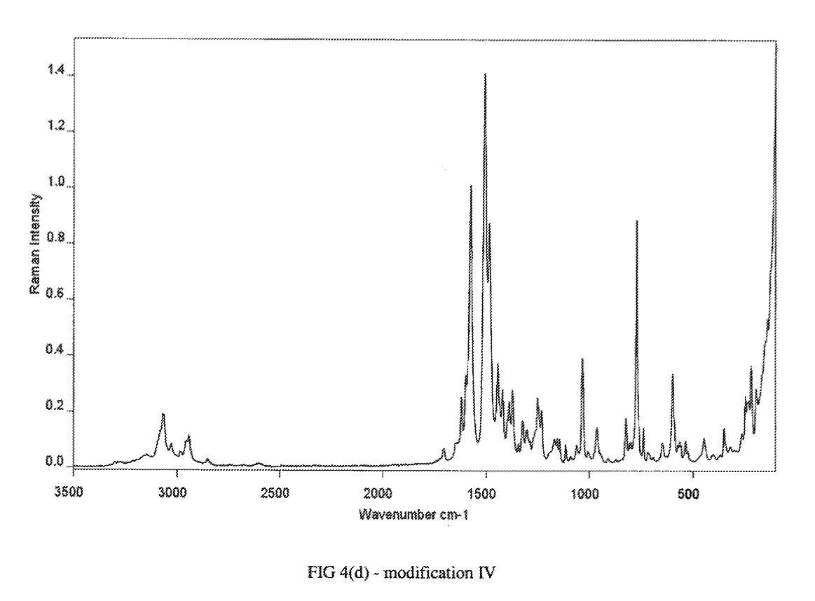

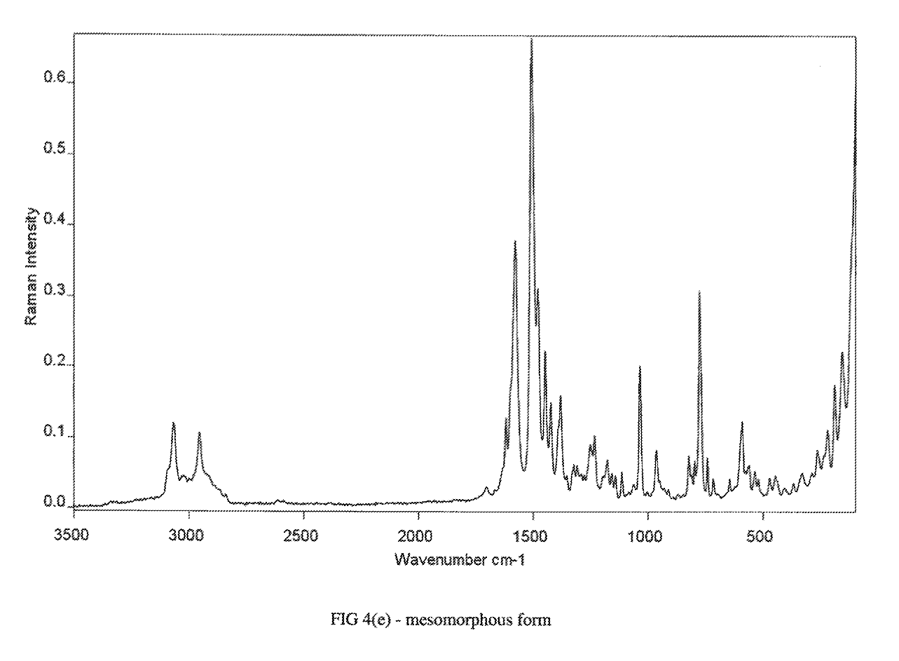

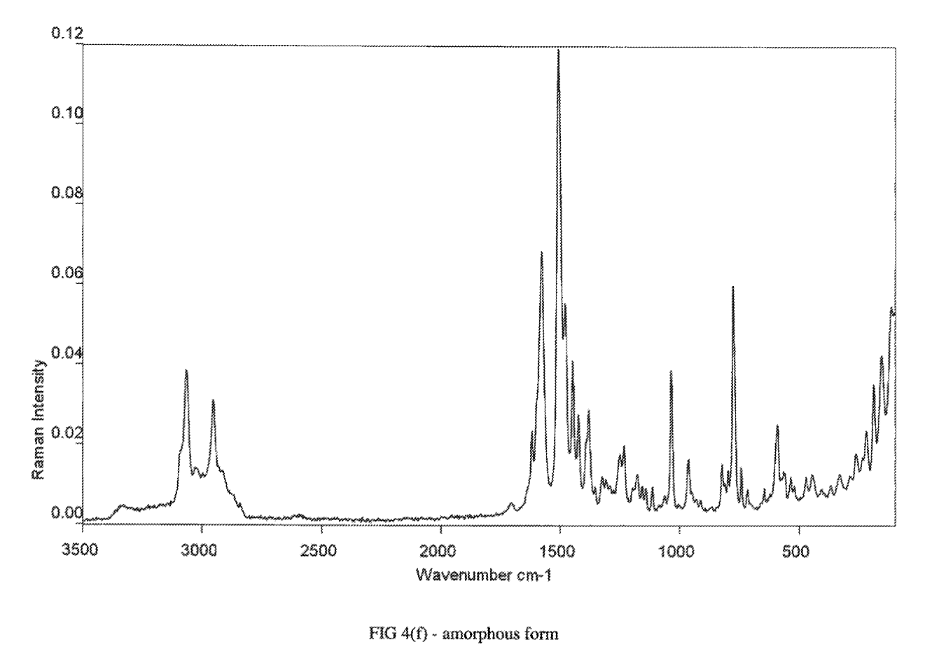

FIG. 4: Raman spectra of modifications I-IV, the mesomorphous form and the amorphous form

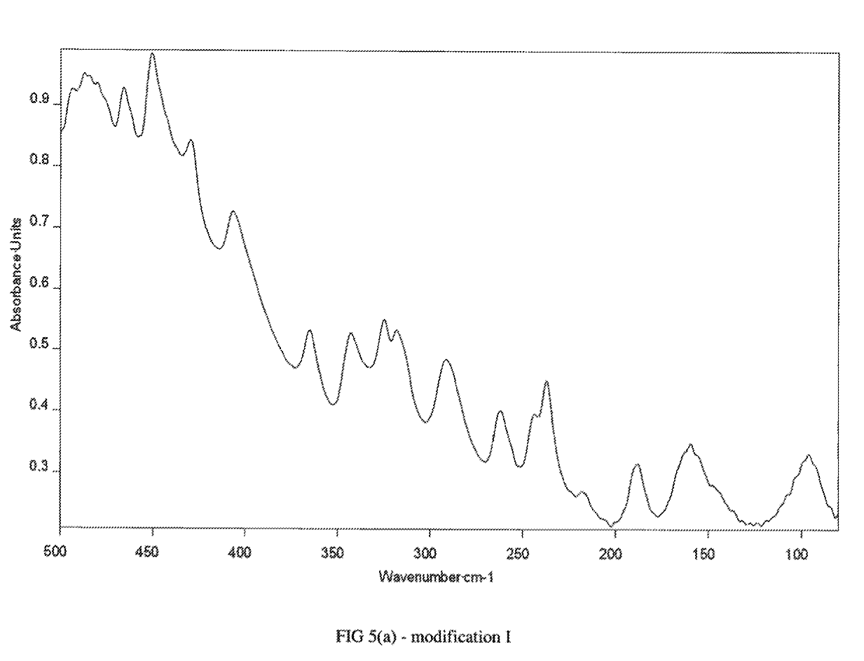

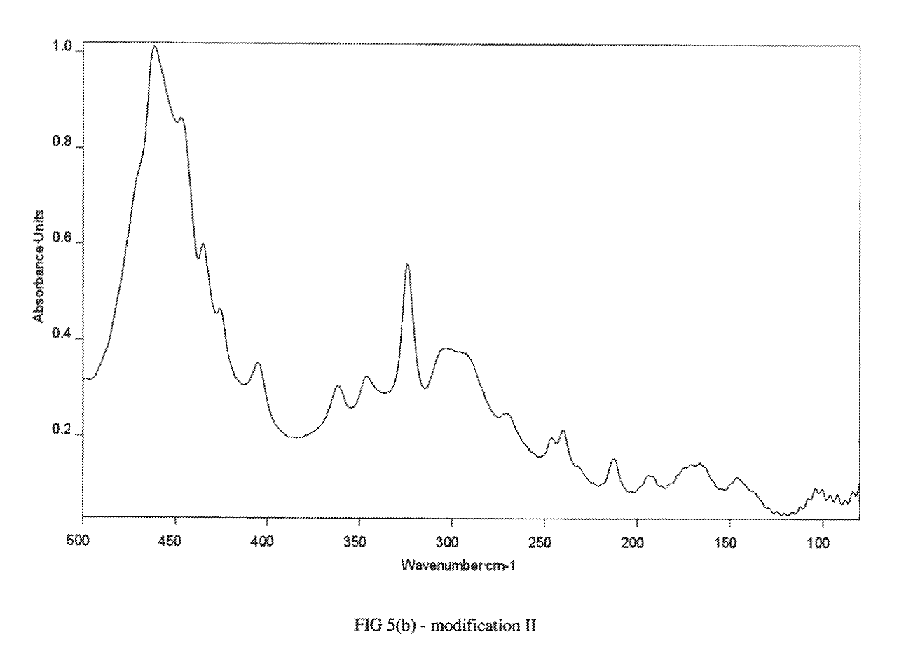

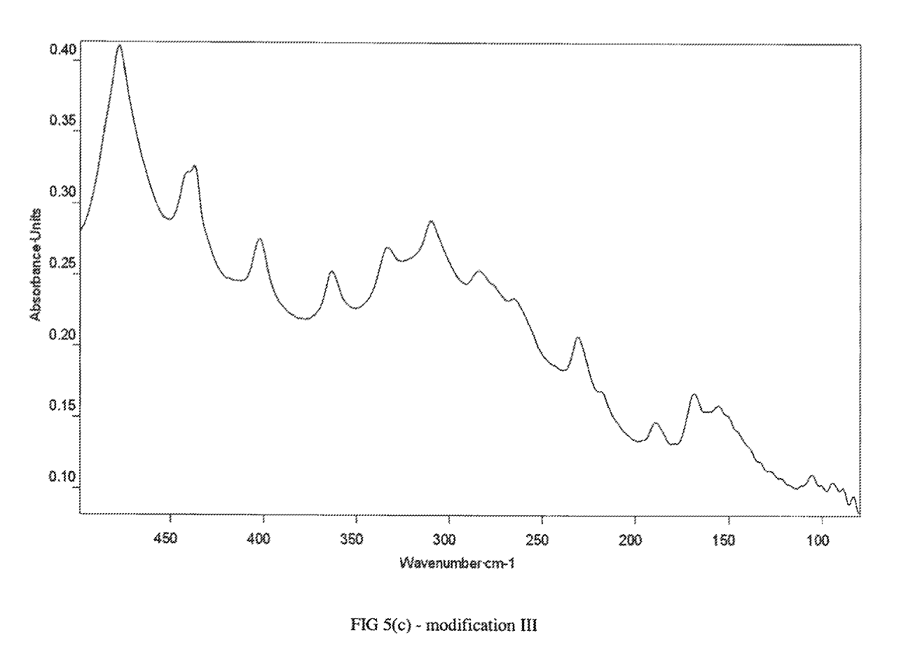

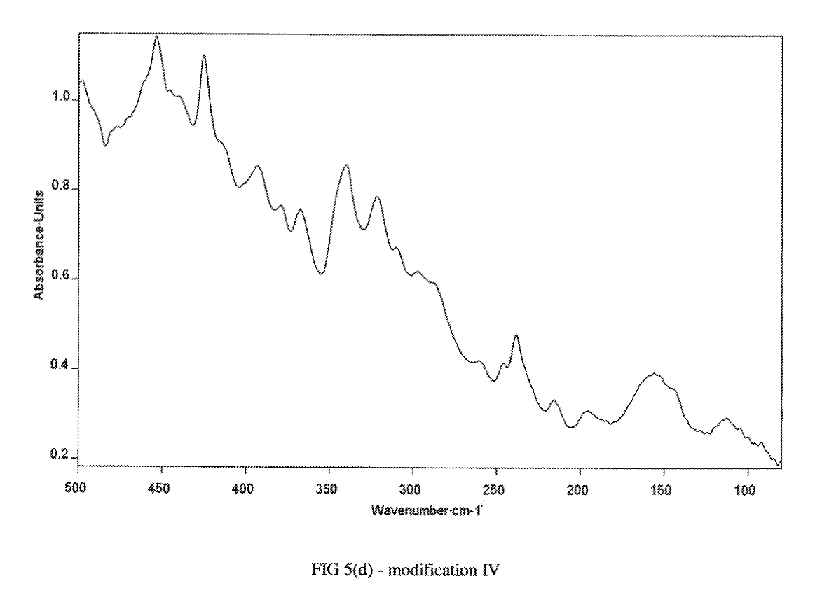

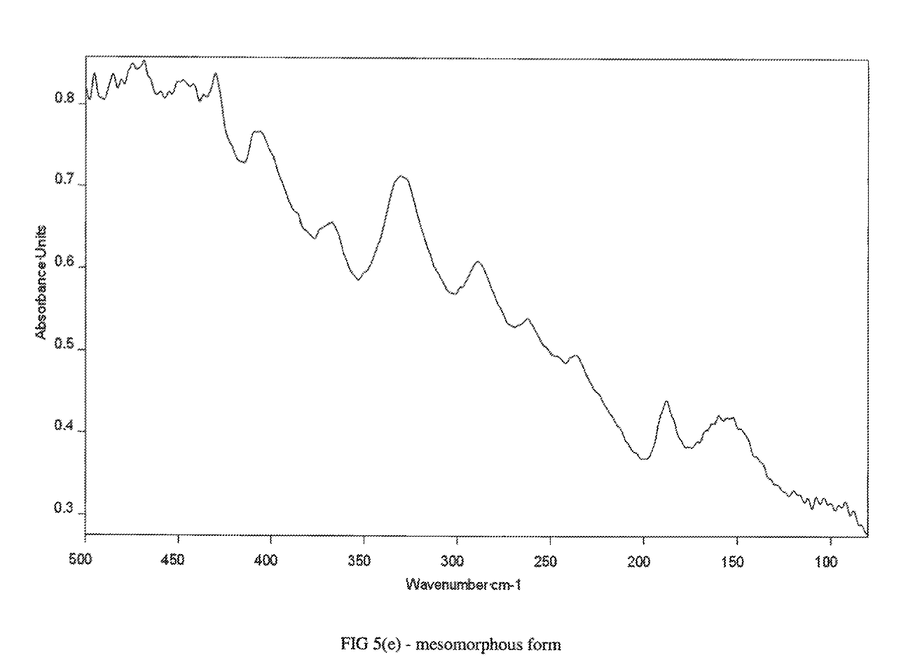

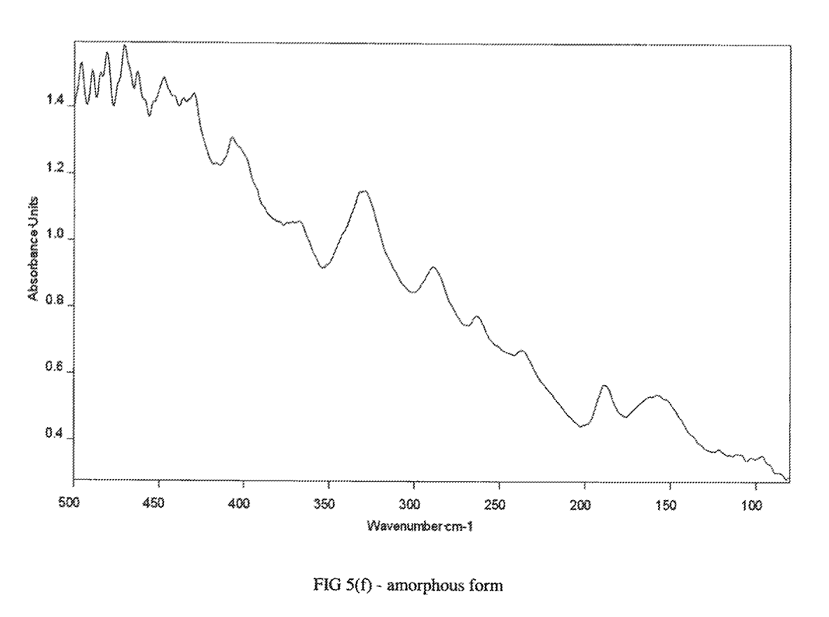

FIG. 5: FIR spectra of modifications I-IV, the mesomorphous form and the amorphous form

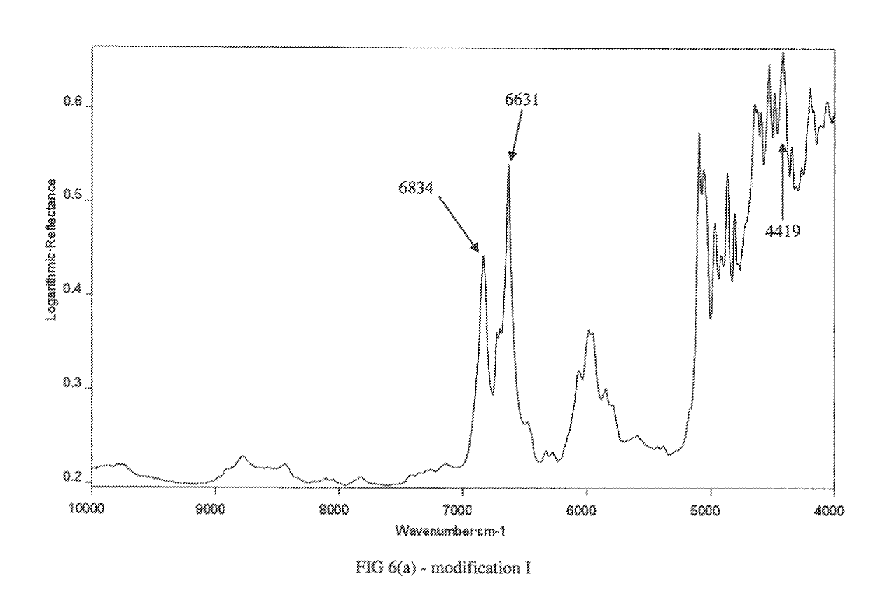

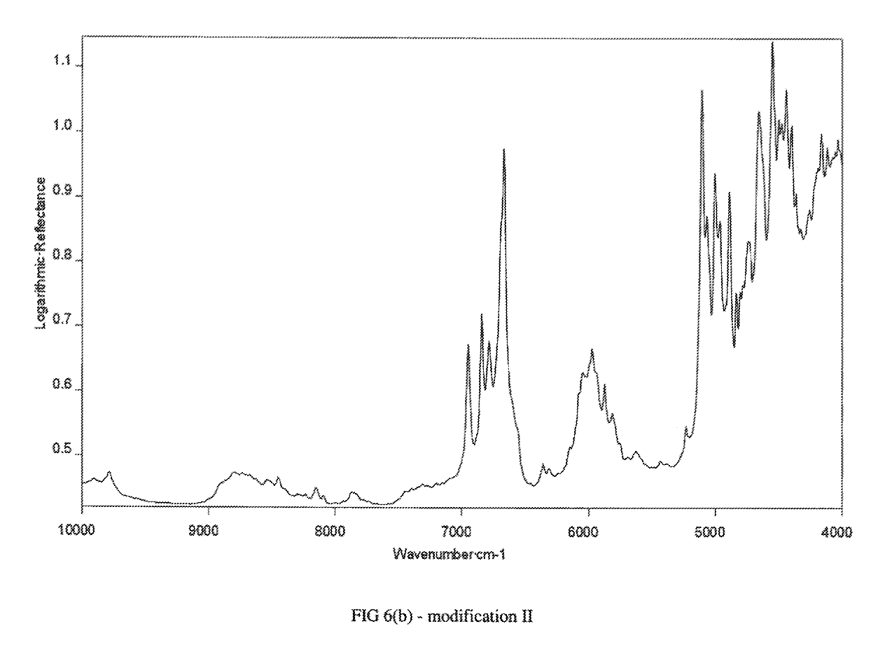

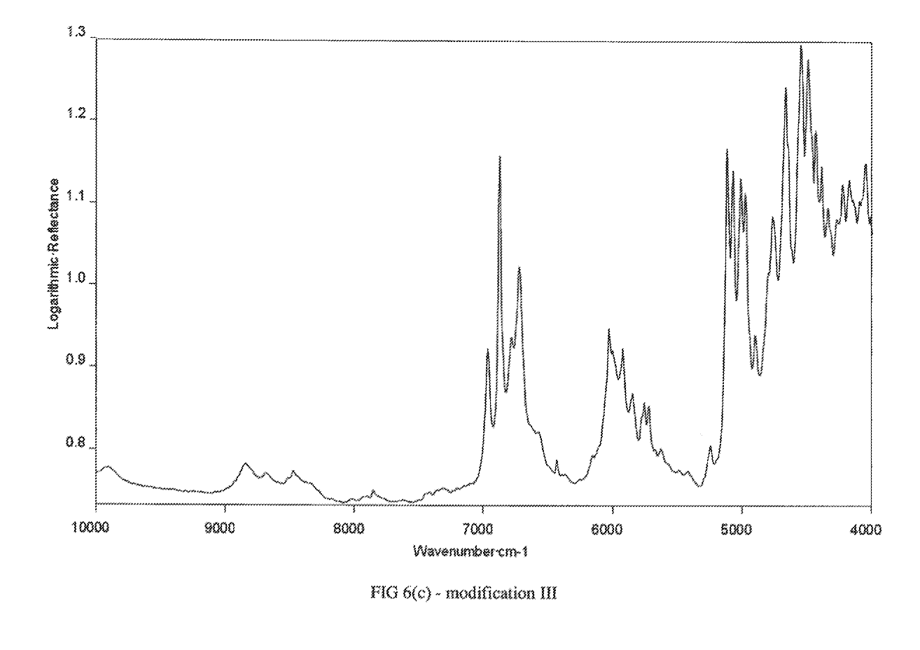

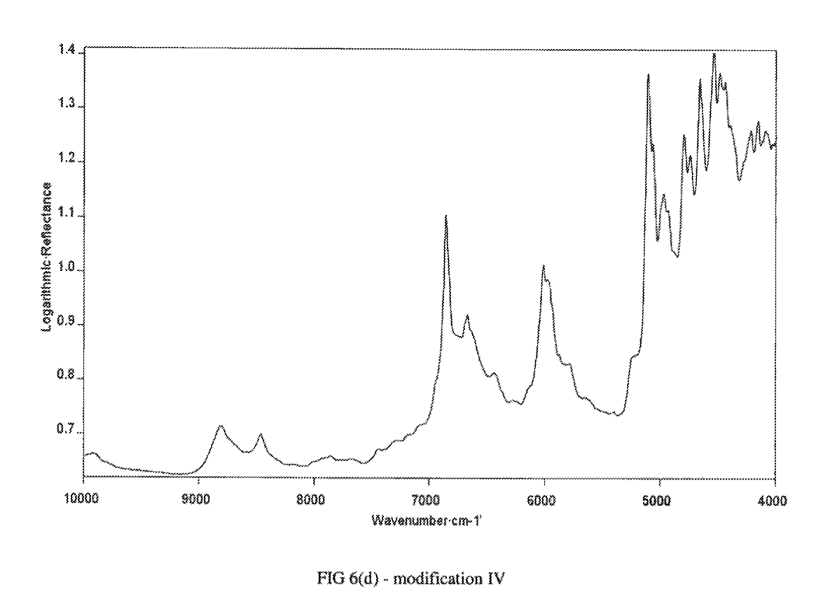

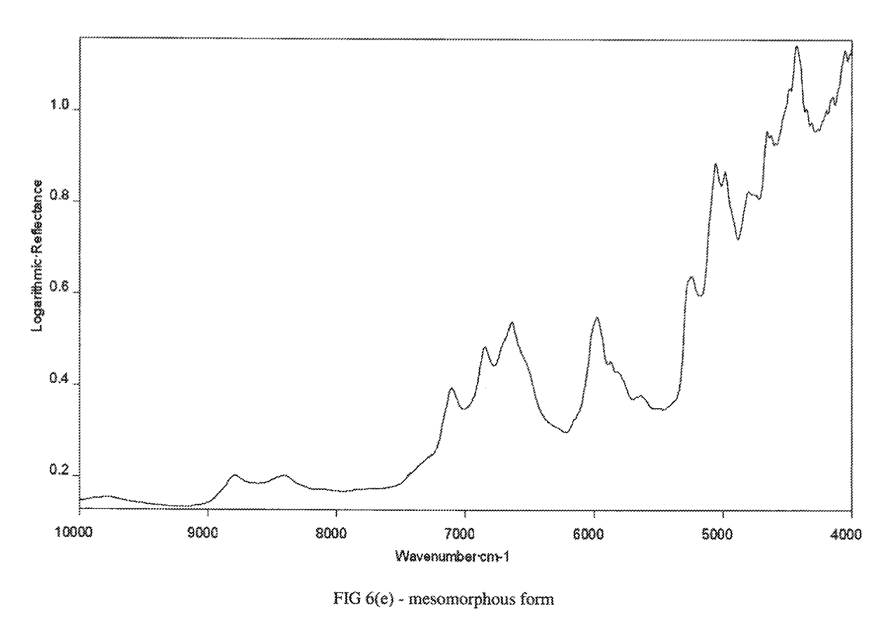

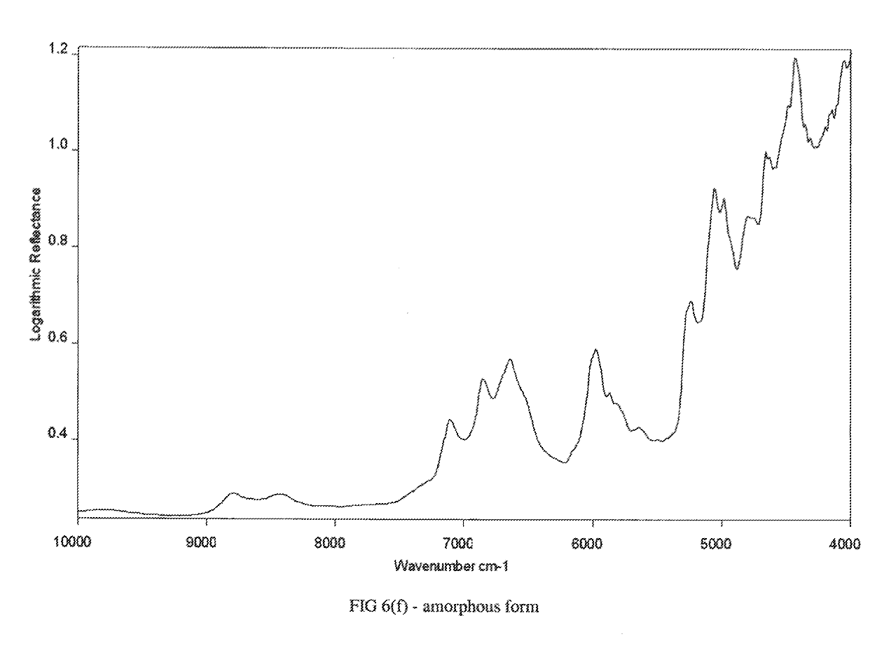

FIG. 6: NIR spectra of modifications I-IV, the mesomorphous form and the amorphous form

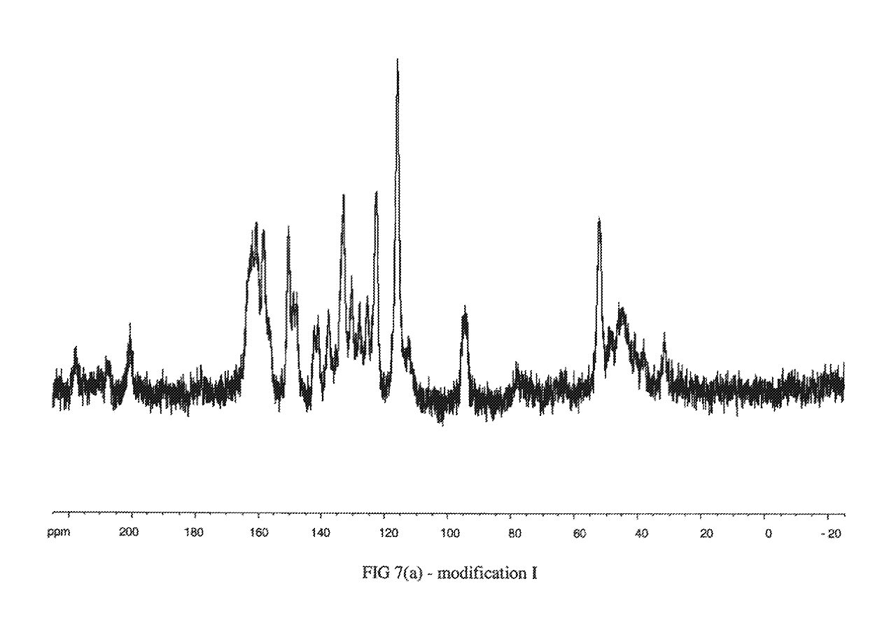

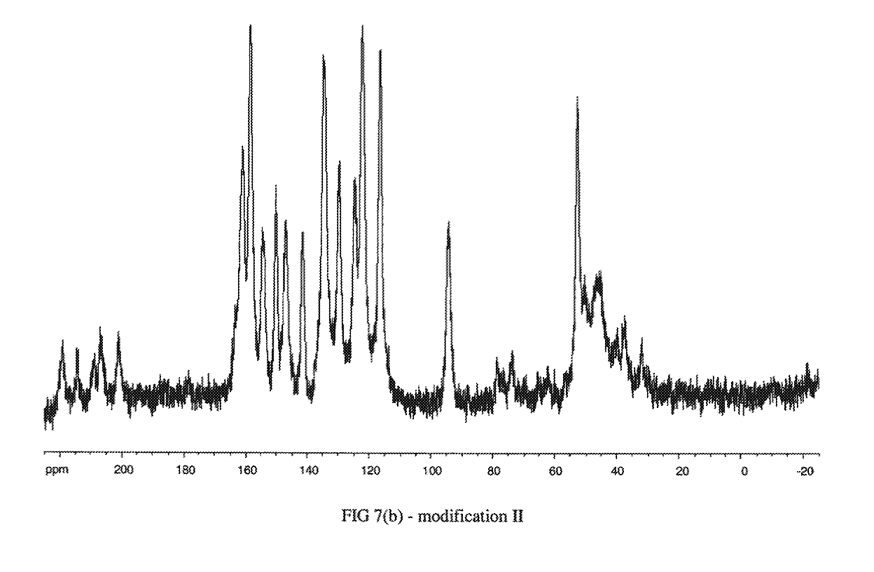

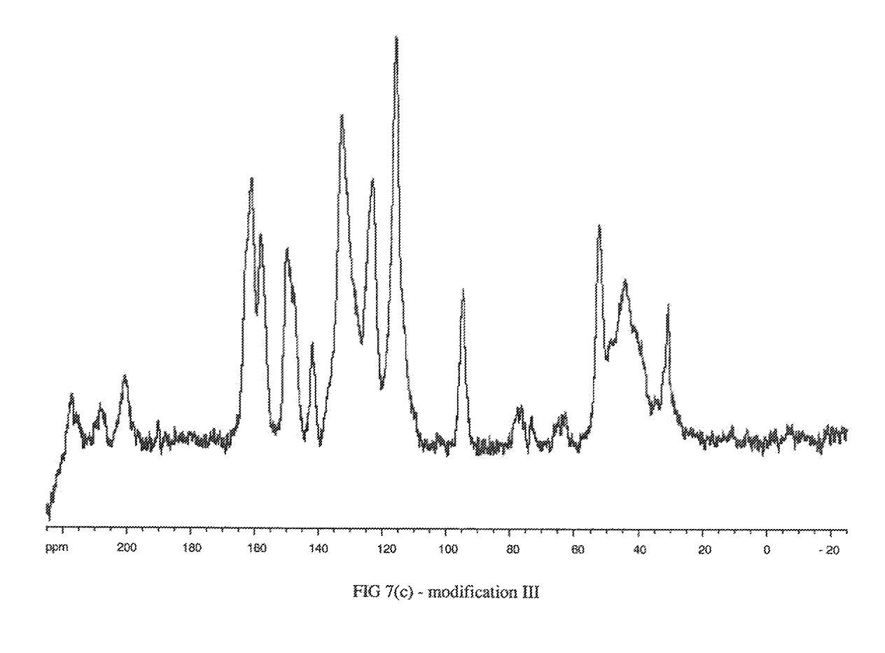

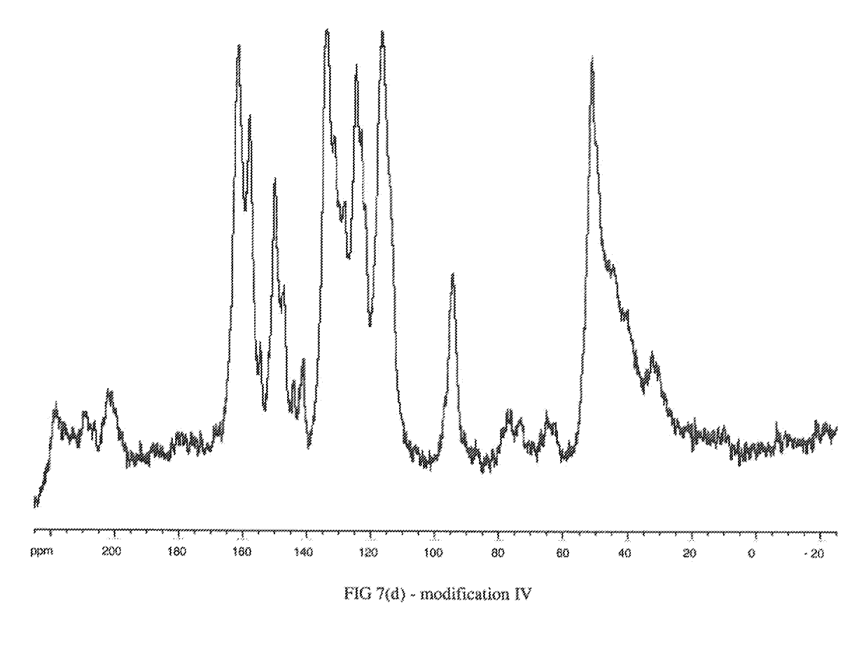

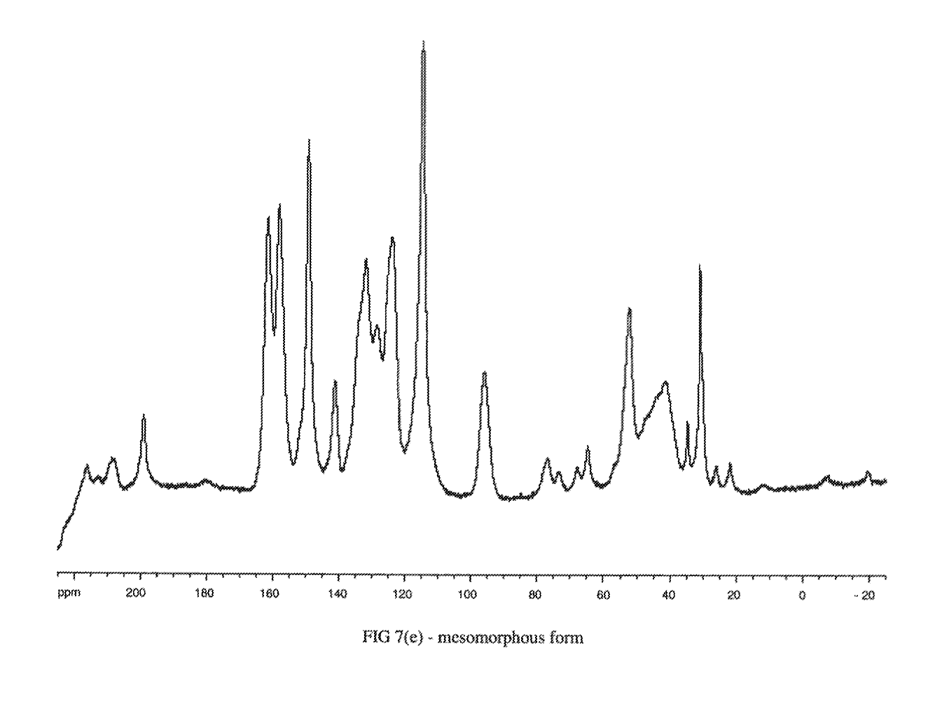

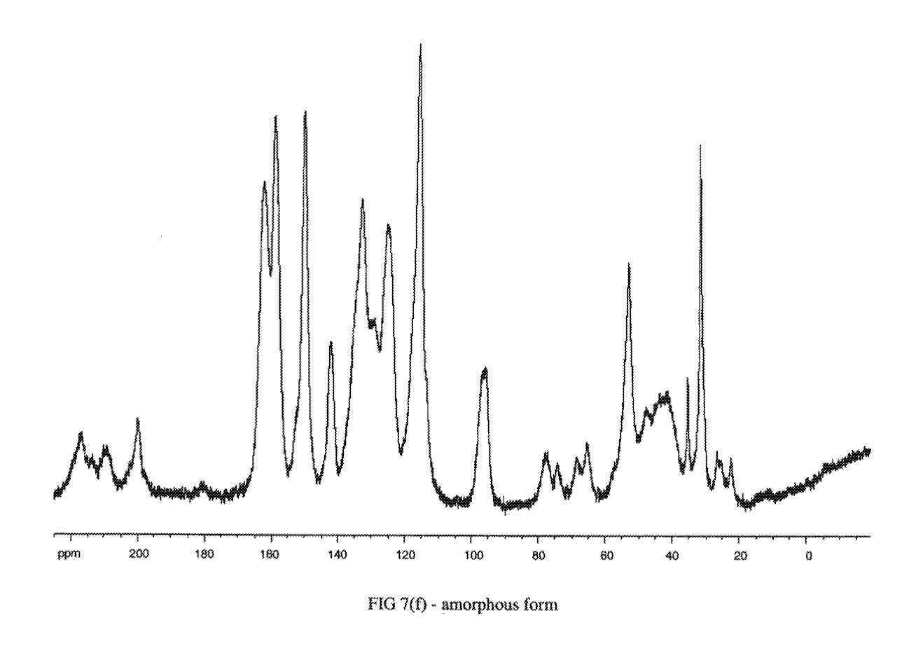

FIG. 7: ¹³C solid-state NMR spectra of modifications I-IV, the mesomorphous form and the amorphous form

1. Methyl {4,6-diamino-2-[1-(2-fluorobenzyl)-1H-pyrazolo[3,4-b]pyridin-3-yl]pyrimidin-5-yl}carbamate of the formula 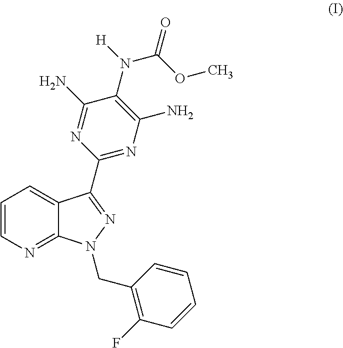
in modification I.

2. The compound according to claim 1, characterized in that the X-ray diffractogram of the compound has a peak maximum of the 2 theta angle at 6.1.

3. The compound according to claim 1, characterized in that X-ray diffractogram of the compound has peak maxima of the 2 theta angle at 6.1, 14.7 and 22.2.

4. The compound according to claim 1, characterized in that the IR spectrum of the compound has a peak maximum at 3451 cm⁻¹.

5. The compound according to claim 1, characterized in that the NIR spectrum of the compound has a peak maximum at 6834 cm⁻¹.

6. The compound according to claim 1, characterized in that the NIR spectrum of the compound has peak maxima at 6834, 6631 and 4419 cm⁻¹.

7. (canceled)

8. A composition comprising a compound according to claim 1 and no major amounts of any other form of the compound of the formula (I).

9. A composition comprising a compound according to claim 1 in an amount of more than 90 percent by weight, based on the total amount of the compound of the formula (I) comprised therein.

10. A process for preparing the compound according to claim 1, comprising suspending the compound of the formula (I) in an inert solvent and stirring or shaking at a temperature of from 10° C. to the reflux temperature of the solvent until quantitative conversion into modification I has been achieved.

11. (canceled)

12. A method for treating a cardiovascular disorder comprising administering to a human or animal in need thereof an effective amount of a compound according to claim 1.
